(19) United States
(12) Patent Application Publication
(10) Pub. No.: US 2007/0258908 A1
(43) Pub. Date: Nov. 8, 2007

(54) Detection and imaging of target tissue

(76) Inventors: Gregory M. Lanza, St. Louis, MO (US); Samuel A. Wickline, St. Louis, MO (US)

Correspondence Address: MORRISON & FOERSTER LLP, 12531 HIGH BLUFF DRIVE, SUITE 100, SAN DIEGO, CA, 92130-2040, US

(21) Appl. No.: 11/796,415

(22) Filed: Apr. 27, 2007

Related U.S. Application Data
(60) Provisional application No. 60/858,065, filed on Nov. 9, 2006.
(60) Provisional application No. 60/795,533, filed on Apr. 27, 2006.

(51) Int. Cl. A61K 49/10

(52) U.S. Cl. 424/9.322; 977/930

(57) ABSTRACT

Methods for high resolution imaging of a suspected target tissue are encompassed by the invention. Such methods include administering low resolution and high resolution contrast agents specific to targeted cells or tissues. The contrast agents are allowed to bind to the target cells or accumulate in a target tissue. A low resolution imaging technique is used to localize an accumulation of the low resolution contrast agent in a target tissue. A high resolution image of the target tissue is then obtained to localize an accumulation of the higher resolution contrast agent, allowing the generation of a higher resolution image than that obtained by the use of the low resolution contrast agent alone. These methods may utilize nanoparticles optionally in an emulsion as a contrast agent.

### CROSS-REFERENCE TO RELATED APPLICATIONS

This application claims the benefit of U.S. Provisional Application Ser. No. 60/858,065, filed Nov. 9, 2006, and U.S. Provisional Application Ser. No. 60/795,533, filed Apr. 27, 2006, the contents of which are incorporated by reference herein in their entirety.

### TECHNICAL FIELD

This invention concerns administering targeted low resolution contrast agents to subjects to provide identification, localization, and low resolution imaging of a target tissue such as a tumor. Simultaneous with this administration or subsequent thereto, a similarly targeted composition that provides higher resolution imaging is provided, such that the administration of the low resolution contrast agent guides the process of high resolution imaging. The invention also relates to the making and administration of emulsions comprising the low and higher resolution contrast agents for imaging.

### BACKGROUND OF THE INVENTION

Data accumulated over the last 25 years in the Surveillance, Epidemiology, and End Results (SEER) cancer registry support the principle that earlier tumor detection improves 5-year survival of patients with either localized or regional invasive breast carcinoma (Elkin et al. (2005) Cancer 104(6):1149-1157). Improvements in survival were correlated with an overall downward shift in tumor size distribution, with particular advantage noted among patients presenting with cancers less than 1 cm. A widespread desire to detect and treat cancer earlier has spawned interest in molecular imaging and genomic-proteomic technologies, which in combination with new strategies to treat cancer, may further improve cancer survival.

One approach to identifying small solid tumors has involved early detection of angiogenesis by targeting unique biosignatures of neovascular endothelium, such as αᵥβ₃-integrin. The inventors have previously demonstrated that paramagnetic perfluorocarbon emulsions targeted to the αᵥβ₃-integrin can be used to detect the neovasculature of tumors 30 mm³ at clinical field strengths (1.5 T). Because perfluorocarbon nanoparticles have a nominal particle size of 250 nm and are constrained within the vasculature, access to αᵥβ₃-integrin expressed on extravascular macrophages, smooth muscle, and other cells is stearically precluded. MRI provides outstanding high-resolution images of even minute tumors enhanced by the bound paramagnetic nanoparticles, as shown in multiple models (Winter et al. (2003) Cancer Res. 63(18):5838-5843; Schnieder et al. (2005) Magn. Reson. Med. 53(3):621-627), but in clinical practice the procedure requires apriori knowledge of the tumor location in order to position coils, establish a field-of-view, and acquire images. Identification of minute tumors in one or more unknown locations may require the high sensitivity of a radionuclide signal such as ¹¹¹In or ^{99m}Tc, which can be detected robustly over a large region-of-interest.

Numerous radiolabeled αᵥβ₃-integrin or vitronectin antagonists, including antibodies, peptides, peptidomimetics, and disintegrins, have been explored as tumor vasculature targeting agents (Haubner et al. (2001) J. Nucl. Med. 42:326-336; Haubner et al. (1999) J. Nucl. Med 40:1061-1071; Janssen et al. (2002) Cancer Res. 62(21):6146-6151; McQuade et al. (2004) Bioconjug. Chem. 15(5):988-996; Chen et al. (2004) Eur. J. Nucl. Med. Mol. Imaging 31(8): 1081-1089; Chen et al. (2004) Nuc. Med Biol. 31(1): 11-19; Chen et al. (2004) Nucl. Med. Biol. 31(2):179-189; Chen et al. (2004) Bioconjug. Chem. 15(1):41-49; Onthank et al. (2004) Bioconjug. Chem. 15(2):235-241; Sadeghi et al. (2004) Circulation 110(1):84-90). Although these agents can be exquisitely specific for αᵥβ₃-integrin, their penetration beyond the circulation allow binding to a cadre of nonendothelial sources. The biodistribution of perfluorocarbon nanoparticles to reticuloendothelial (RES) organs is well known and previously reported (McGoron et al. (1994) Artif. Cells Blood Substit. Immobil. Biotechnol. 22:1243-1250), but the potential for higher radionuclide payloads and their intravascular distribution make them attractive agents for rapid identification of nascent tumors in nonRES tissues, including the head, neck, lung, abdomen, pelvis, and bones.

There remains a continuing need for developing approaches and compositions that are useful for reaching a variety and/or particular sites and tissues within an individual and that result in an enhanced degree of contrast, specificity and sensitivity for molecular imaging and therapeutic agent delivery.

All publications, patent applications, and patents cited herein are hereby incorporated by reference in their entirety.

### BRIEF SUMMARY OF THE INVENTION

The invention provides compositions which are liquid emulsions. The liquid emulsions contain nanoparticles comprised of liquid, relatively high boiling perfluorocarbons surrounded by a coating which is composed of a lipid and/or surfactant. The surrounding coating is able to couple directly to a moiety that targets αᵥβ₃ or can entrap an intermediate component which is covalently coupled to the said moiety, optionally through a linker. Alternatively, the coating may be cationic so that negatively charged αᵥβ₃ targeting agents such as nucleic acids, in general or aptamers, in particular, can be adsorbed to the surface.

The compositions of the invention are intended to target tissues expressing the target moiety, and such targeting is intended to be detected using low resolution and higher resolution imaging techniques. In one embodiment, the low resolution contrast agent comprises a radionuclide or optical imaging agent, which can be coupled to a target-specific ligand. Optionally, the low resolution contrast agent comprises a particle, such as a nanoparticle. Other types of particles include liposomes, micelles, bubbles containing gas and/or gas precursors, lipoproteins, halocarbon and/or hydrocarbon nanoparticles, halocarbon and/or hydrocarbon emulsion droplets, hollow and/or porous particles and/or solid nanoparticles. In one embodiment, the low resolution contrast agent comprises a halocarbon-based nanoparticle such as a perfluorooctyl bromide (PFOB) nanoparticle, detectable, for example, with fluorine MRI. A higher resolution contrast agent comprises a target-specific ligand, a contrast agent for magnetic resonance imaging (MRI), a CT imaging agent, an optical imaging agent, an ultrasound imaging agent, a paraCEST imaging agent, or a combination thereof, and, optionally, comprises a particle such as a nanoparticle. The low resolution and higher resolution contrast agent can be incorporated into the same particle.

A targeted low resolution contrast agent accumulates in tissues expressing the target moiety. A low resolution imaging technique identifies potential target tissues that contain an accumulation of the low resolution contrast agent. A targeted higher resolution contrast agent is administered having an analogous target as the low resolution contrast agent, which will also accumulate in the potential target tissue. If any potential target tissue is identified using the low resolution imaging technique, a higher resolution imaging technique is used to examine any identified potential target tissues at a higher resolution.

Thus, in one aspect, the invention is directed to a method for high resolution imaging, comprising: (a) administering a targeted low resolution contrast agent and a targeted higher resolution contrast agent having an analogous target as the low resolution contrast agent, and allowing each contrast agent to accumulate in a target tissue; (b) identifying the target tissue using a low resolution imaging technique to localize an accumulation of the low resolution contrast agent. If the low resolution imaging technique identifies a target tissue having an accumulation of the low resolution contrast agent, step (c) is applied, directed to obtaining a high resolution image of the target tissue using a higher resolution imaging technique to localize an accumulation of the higher resolution contrast agent, thereby allowing the generation of a higher resolution image than that obtained by the use of the low resolution contrast agent alone.

In another aspect, the invention is also directed to a method of delivering targeted contrast agents to a target tissue, comprising: (a) administering a low resolution targeted contrast agent selected from a targeted nuclear contrast agent and a halocarbon-based nanoparticle to a subject comprising said target tissue; (b) administering a higher resolution targeted contrast agent to the subject, selected from the group consisting of an MRI contrast agent, a CT contrast agent, an ultrasound contrast agent, an optical contrast agent, a paraCEST contrast agent and a combination thereof, wherein the higher resolution contrast agent has an analogous target as the low resolution contrast agent; and (c) allowing the contrast agents to accumulate in the target tissue, to thereby deliver targeted contrast agents to the target tissue. An image of the low resolution contrast agent that is bound to the targeted tissue can be obtained. In another embodiment, an image of the higher resolution contrast agent that is bound to the targeted tissue is obtained, optionally after the image of the low resolution contrast agent bound to the targeted tissue is obtained.

### BRIEF DESCRIPTION OF THE DRAWINGS

FIG. 1A shows a pharmacokinetic profile depicting the distribution and clearance from circulation of αᵥβ₃-targeted ˜10 ¹¹¹In nanoparticles (NP) with ˜10 ¹¹¹In/NP. Percent injected dose (ID) in blood versus time post injection is presented for one animal over the initial two hours. A two-compartment bi-exponential model was applied to the data from each animal. Estimates of beta elimination half-life, volume of distribution and clearance were calculated and are presented as a mean±SD, n=6. FIG. 1B shows the biodistribution of perfluorocarbon nanoparticles in rabbits injected with nanoparticle emulsion at dosages of 0.25 ml/kg, 0.5 ml/kg, and 1.0 ml/kg (n=3/dose). Tissue perfluorocarbon content was measured directly by gas chromatography and results are presented as % ID/g±SD tissue.

FIGS. 2A, B, and C show the ratio of tumor-to-muscle signal. The ratio of tumor-to-muscle signal was determined immediately after contrast injection and serially every 15 minutes in rabbits implanted with Vx-2 after receiving 22 MBq/kg (i.v.) of: A) αᵥβ₃-targeted ¹¹¹In nanoparticles (NP) with ˜10 ¹¹¹In/NP versus αᵥβ₃-targeted nonlabeled (Competition); B) αᵥβ₃-targeted or nontargeted ¹¹¹In nanoparticles with ˜10 ¹¹¹In/NP and, C) αᵥβ₃-targeted ¹¹¹In nanoparticles with ˜10 ¹¹¹In/NP versus ˜1 ¹¹¹In/NP. Values presented represent the mean±SEM; * p<0.05 over 2 hr.

FIGS. 3A and B show the 18 hour ¹¹¹In planar image (15 minute scan, 128×128 matrix) of rabbits implanted ˜12 days previously with Vx-2 tumor following 22 MBq/kg (i.v.) of nontargeted (A) or αᵥβ₃-targeted (B) ¹¹¹In nanoparticles (NP) bearing ˜10 ¹¹¹In/NP.

FIG. 4A shows a microscopic image (4×) of Vx-2 adenocarcinoma adjacent to muscle and stained for αᵥβ₃-integrin, which appear as dark brown (purple) streaks (white arrows) within the intervening connective tissue. FIGS. 4B and C show higher magnification regions (20×) of relatively sparse (B) and dense regions (C) of αᵥβ₃-integrin positive neovessels identified on primary image.

FIG. 5A shows a microscopic image (4×) of Vx-2 adenocarcinoma stained for RAM 11, a biomarker specific for macrophages, which appear as dark brown (purple) accumulations dispersed within the core of the tumor but less prevalent in the peripheral capsule. FIG. 5B is an enlarged view of A revealing macrophage distribution within the core of the tumor (white arrows).

FIG. 6A shows a light microscopic image (4×) of Vx-2 adenocarcinoma and capsule. Note necrosis towards the center and cellular proliferation occurring around the periphery of the tumor. FIG. 6B shows a fluorescent microscopy image (20×) of tumor capsule region depicted in A. The green signature of vessels retaining αᵥβ₃-integrin targeted AlexaFluor 488 nanoparticles within the capsule (arrows). Blue DAPI staining represents cellular nuclei within the connective tissue.

FIGS. 7A-C show fluorescent microscopy images (40×) of αᵥβ₃-integrin targeted rhodamine nanoparticles (B) and FITC-lectin (A) and the merged images obtained from the tumor capsule region (C). Note the αᵥβ₃-integrin targeted rhodamine nanoparticles and the FITC-lectin are spatially co-localized as shown in (C). Rhodamine nanoparticles were not found in the extravascular spaces of the tumor or capsule.

### MODES OF CARRYING OUT THE INVENTION

The present invention offers a kit for the preparation of an emulsion of particles such as nanoparticles targeted to tissue expressing a target moiety, which kit comprises at least one container that contains nanoparticles comprising a ligand specific for the target moiety and a linking moiety for coupling to a low resolution contrast agent and/or a higher resolution contrast agent, at least one container that contains said low resolution contrast agent, and at least one container that contains said higher resolution contrast agent. In one embodiment, the target moiety is αᵥβ₃.

Also encompassed are kits for the preparation of an emulsion of nanoparticles targeted to tissue expressing a target moiety, which kit comprises at least one container that contains nanoparticles comprising a linking moiety for coupling to a ligand specific for the target moiety, at least one container that contains a ligand specific for the target moiety, at least one container that contains a low resolution contrast agent, and at least one container that contains a higher resolution contrast agent. In one embodiment, the target moiety is αᵥβ₃.

The nanoparticles for use in the invention can be high-boiling liquid perfluorocarbon-based nanoparticles that further comprise a coating of lipid/surfactant. As described in further detail below, a target-specific ligand, which in certain embodiments is a αᵥβ₃-specific ligand, can be coupled covalently to a component of the lipid/surfactant coating.

Additionally, the invention is directed to a kit for high resolution imaging, comprising at least one container that contains a targeted low resolution contrast agent, at least one container that contains a targeted higher resolution contrast agent, and instruction means for use. One or both of the contrast agents can comprise particles, such as, but not limited to, nanoparticles. In one embodiment, the kit comprises at least one container that contains nanoparticles comprising a ligand specific for a target moiety coupled via a linking moiety to a low resolution contrast agent, and at least one container that contains nanoparticles comprising a ligand specific for the target moiety coupled via a linking moiety to a higher resolution contrast agent. In another embodiment, the kit comprises at least one container containing halocarbon-based nanoparticles comprising a ligand specific for a target moiety and a higher resolution contrast agent, such that both the low resolution and higher resolution contrast agents are incorporated into the same nanoparticle. The halocarbon-based nanoparticle may be detectable using a low resolution imaging technique. Such nanoparticles can be detected, for example, using fluorine MRI as the low resolution imaging technique. In one embodiment, the nanoparticles are administered to a subject, and a low resolution imaging technique is employed to identify a target tissue in the subject. In a further embodiment, a higher resolution imaging technique is then used to obtain an image of the target tissue. In one embodiment, the target moiety is αᵥβ₃.

In another embodiment, the invention is directed to a kit for high resolution imaging, comprising at least one container that contains halocarbon-based nanoparticles comprising a ligand specific for a target moiety, wherein the nanoparticles are coupled to a higher resolution contrast agent, and instruction means for use. The halocarbon-based nanoparticles can comprise perfluorooctylbromide (PFOB). In one embodiment, the higher resolution contrast agent comprises a MRI contrast agent. In a method for obtaining a high resolution image of a target tissue, the composition is administered to a subject, a target tissue is identified using fluorine MRI to localize an accumulation of the low resolution contrast agent, and an MRI image of the target tissue is obtained, thus generating a high resolution image of the target tissue.

The invention further encompasses a method for high resolution imaging, comprising: (a) administering a targeted low resolution contrast agent and a targeted higher resolution contrast agent having an analogous target as the low resolution contrast agent to a subject, and allowing each contrast agent to accumulate in one or more target tissues; (b) using a low resolution imaging technique to localize an accumulation of the low resolution contrast agent in a target tissue; and (c) obtaining a high resolution image of the target tissue using a higher resolution imaging technique to localize an accumulation of the higher resolution contrast agent, thereby allowing the generation of a higher resolution image than that obtained by the use of the low resolution contrast agent alone. The target tissue can be contained within a mammalian subject, and is preferably contained in a human subject. The low resolution contrast agent and the higher resolution contrast agent can be incorporated into the same composition, which is detectable using a low resolution modality and a higher resolution modality. For example, the agent can be a gadolinium-loaded perfluorocarbon emulsion, initially detectable via fluorine MRI as the low resolution imaging technique and detectable using proton MRI as a higher resolution imaging technique. In one embodiment, the low resolution contrast agent and higher resolution contrast agent are incorporated into a particle such as a nanoparticle as described further herein.

A decoy particle can be administered simultaneously with the low resolution contrast agent. Decoy particles are described, for example, in PCT Publication No. WO 05/086639.

The low resolution contrast agent can be administered simultaneously with the higher resolution contrast agent. In one embodiment, the low resolution and higher resolution contrast agents are incorporated into the same nanoparticle. Alternatively, the higher resolution contrast targeting agent is administered subsequent to the low resolution contrast agent.

The invention is also directed to a method of delivering targeted contrast agents to a target tissue, comprising: (a) administering a low resolution targeted contrast agent to a subject containing a suspected target tissue; (b) administering a higher resolution targeted contrast agent to the subject, wherein the higher resolution contrast agent has an analogous target as the low resolution contrast agent; and (c) allowing the contrast agents to accumulate in the target tissue, to thereby deliver targeted contrast agents to the target tissue. An image of the low resolution contrast agent that is bound to the targeted tissue can be obtained. In another embodiment, an image of the higher resolution contrast agent that is bound to the targeted tissue is obtained, optionally after the image of the low resolution contrast agent bound to the targeted tissue is obtained.

In one embodiment of the invention, the low resolution contrast agent comprises a diagnostic radionuclide and a target ligand. In another embodiment, the low resolution contrast agent comprises a halocarbon-based nanoparticle, such as PFOB or other fluorine-based MRI agents.

In a further embodiment, the higher resolution contrast agent is selected from the group consisting of an MRI agent, a CT imaging agent, an optical imaging agent, an ultrasound imaging agent, a paraCEST imaging agent, and a combination thereof. In another embodiment, the higher resolution contrast agent comprises an MRI agent, which can be fluorine-based, such as PFOB. Alternatively, the higher resolution contrast agent is a proton based MRI or paraCEST agent comprising a chelate of a paramagnetic metal selected from the group consisting of scandium, titanium, vanadium, chromium, manganese, iron, cobalt, nickel, copper, molybdenum, ruthenium, cerium, indium, praseodymium, neodymium, promethium, samarium, europium, gadolinium, terbium, dysprosium, holmium, erbium, thulium, and ytterbium. In a further embodiment, the higher resolution contrast agent can comprise a CT imaging agent comprising an iodinated oil nanoparticles or an entrapped solid metal particle.

The low or higher resolution contrast agent can be incorporated into a vehicle comprising a particle. “Particles” include, for example, liposomes, micelles, bubbles containing gas and/or gas precursors, lipoproteins, halocarbon and/or hydrocarbon nanoparticles, halocarbon and/or hydrocarbon emulsion droplets, hollow and/or porous particles and/or solid nanoparticles. The particles themselves may be of various physical states, including solid particles, solid particles coated with liquid, liquid particles coated with liquid, and gas particles coated with solid or liquid. Various particles useful in the invention have been described in the art as well as means for coupling targeting components to those particles in the active composition. Such particles are described, for example, in U.S. Pat. Nos. 6,548,046; 6,821,506; 5,149,319; 5,542,935; 5,585,112; 5,149,319; 5,922,304; and European publication 727,225, all incorporated herein by reference with respect to the structure of the particles. These documents are merely exemplary and not all-inclusive of the various kinds of particulate vehicles that are useful in the invention. While nanoparticles are generally described herein, it is understood that the embodiments of the invention are not limited to nanoparticles, and that the compositions and methods described herein are similarly useful for other types of particles.

Further, the particles used as vehicles may contain bubbles of gas or precursors which form bubbles of gas when in use. In these cases, the gas is contained in a liquid or solid based coating.

Other suitable particles which may be provided with targeting agents and optionally activity components or used in the carrier include the oil and water emulsions described in U.S. Pat. No. 5,536,489, liposome compositions such as those described in U.S. Pat. No. 5,512,294 and oil and water emulsions as described in U.S. Pat. No. 5,171,737.

In one embodiment, the contrast agent is incorporated into a nanoparticle that can be in an emulsion, as described further herein. Preferably, the nanoparticle comprises a liquid fluorocarbon core surrounded by a lipid coating.

The contrast agent is targeted by a target-specific ligand. In preferred embodiments, the target-specific ligand is an antibody, an antibody fragment, a peptide, an aptamer, a peptide mimetic, a drug or a hormone. The target-specific ligand can be coupled to a nanoparticle. In one embodiment, the target tissue is characterized by high levels of αᵥβ₃ integrin, and in further embodiments, the low resolution and/or high resolution contrast agent comprises an emulsion comprising nanoparticles linked to a ligand for αᵥβ₃ integrin.

In general, the targeted nanoparticles, directly coupled to a target-specific ligand, are useful themselves for X-ray imaging (e.g., computed tomography (CT)), ultrasound imaging and/or delivery of a therapeutic agent. However, the inclusion of other components renders them useful for other forms of imaging, such as, magnetic resonance imaging (MRI), nuclear imaging (e.g., scintigraphy, positron emission tomography (PET) and single photon emission computed tomography (SPECT)), optical or light imaging (e.g., confocal microscopy and fluorescence imaging), magnetotomography and electrical impedance imaging. For instance, the inclusion of a chelating agent containing a paramagnetic ion makes the particle useful as a magnetic resonance imaging contrast agent. Because perfluorocarbon nanoparticles comprise large amounts of fluorine, the addition of a paramagnetic ion is not necessary to make these particles useful for MRI; the fluorocarbon core allows ¹⁹F magnetic resonance imaging to be used to track the location of the particles. ¹⁹F magnetic resonance imaging can be used as the low or higher resolution imagining technique, depending on the nature of the other imaging modality. Additionally, the inclusion of a radionuclide makes an agent useful for nuclear imaging (e.g., scintigraphy, positron emission tomography (PET) and single photon emission computed tomography (SPECT)) or a therapeutic for radiation treatment, or both. The inclusion of biologically active materials makes an agent useful as drug delivery systems. A multiplicity of such activities may be included; thus, images can be obtained of targeted tissues at the same time active therapeutic substances are delivered to them.

Emulsions of halocarbon-based nanoparticles can be prepared in a range of methods depending on the nature of the components to be included in the coating. In a typical procedure, used for illustrative purposes only, the following procedure is set forth: Perfluorooctylbromide (40% w/v, PFOB, 3M), and a surfactant co-mixture (2.0%, w/v) and glycerin (1.7%, w/v) is prepared where the surfactant co-mixture includes 64 mole % lecithin (Pharmacia Inc), 35 mole % cholesterol (Sigma Chemical Co.) and 1 mole % dipalmitoyl-L-alpha-phosphatidyl-ethanolamine, Pierce Inc.) dissolved in chloroform. A drug is suspended in methanol (˜25 μg/20 μl) and added in titrated amounts between 0.01 and 5.0 mole % of the 2% surfactant layer, preferably between 0.2 and 2.0 mole %. The chloroform-lipid mixture is evaporated under reduced pressure, dried in a 50° C. vacuum oven overnight and dispersed into water by sonication. The suspension is transferred into a blender cup (Dynamics Corporation of America) with perfluorooctylbromide in distilled or deionized water and emulsified for 30 to 60 seconds. The emulsified mixture is transferred to a Microfluidics emulsifier (Microfluidics Co.) and continuously processed at 20,000 PSI for three minutes. The completed emulsion is vialed, blanketed with nitrogen and sealed with stopper crimp seal until use. A control emulsion can be prepared identically excluding the drug from the surfactant commixture. Particle sizes are determined in triplicate at 37° C. with a laser light scattering submicron particle size analyzer (Malvern Zetasizer 4, Malvern Instruments Ltd., Southborough, Mass.), which indicate tight and highly reproducible size distribution with average diameters less than 400 nm. Unincorporated drug can be removed by dialysis or ultrafiltration techniques. To provide the targeting ligand, an F(ab) fragment is coupled covalently to the phosphatidyl ethanolamine through a bifunctional linker in the procedure described above.

In some instances, the lipid and/or surfactant surrounding coating is able to couple directly to a targeting moiety or can entrap an intermediate component which is covalently coupled to the targeting moiety, optionally through a linker, or may contain a non-specific coupling agent such as biotin. Alternatively, the coating may be cationic or anionic so that targeting agents can be electrostatically adsorbed to the surface. For example, the coating may be cationic so that negatively charged targeting agents such as nucleic acids, in general, or aptamers, in particular, can be adsorbed to the surface.

In some embodiments, the nanoparticles may contain associated with their surface at least one “ancillary agent” useful in imaging and/or therapy including, but not limited to, a radionuclide, a contrast agent for MRI or for PET imaging, a fluorophore or infrared agent for optical imaging, and/or a biologically active compound. The nanoparticles themselves can serve as contrast agents for X-ray (e.g., CT), fluorine-based MRI, or ultrasound imaging. In other embodiments, the nanoparticle is linked to a low resolution and higher resolution contrast agent, each of which may be further associated with one or more ancillary agents.

In some embodiments, the contrast agents may be modified to incorporate therapeutic agents including, but not limited to, bioactive, radioactive, chemotherapeutic and/or genetic agents, for use as a therapeutic agent as well as a diagnostic agent.

The invention also provides methods of using the contrast agents in a variety of applications including in vivo, ex vivo, in situ and in vitro applications. The methods include single- or multi-modal imaging and/or therapy methods.

Thus, targeted contrast agents that incorporate at least one therapeutic agent are particularly useful for the treatment of a disease or disorder that has improved risk/benefit profiles when applied specifically to selected cells, tissues and/or organs.

### Methods of use and Compositions of the Invention

The emulsions and kits for their preparation are useful in the methods of the invention which include imaging of cells, tissues and/or organs, and/or delivery of therapeutic agents to the cells, tissues and/or organs. In some embodiments, the emulsions are targeted to a particular cell type and/or tissue through the use of ligands directed to the cell and/or tissue on the surface of the emulsions. The emulsions can be used with cells or tissues in vivo, ex vivo, in situ and in vitro.

In vitro or ex vivo use of the emulsions containing a targeting ligand and an agent (e.g., drug) can, for example, identify and/or deliver the agent to the targeted cell. Such cells can be identified using X-ray imaging techniques, for example, and agent delivery to the cell can also be confirmed through the imaging process. For example, the targeted emulsions can be used to deliver genetic material to cells, e.g., stem cells, and/or to label cells, e.g., stem cells, ex vivo or in vitro before implantation or further use of the cells. Additionally, the emulsions of the invention can be used to identify targeted cells in solution and to collect or isolate targeted cells from a solution, for example, by precipitation and/or gradient centrifugation.

The methods of using the nanoparticulate emulsions of the invention in vivo and in vitro are well known to those in the art. Cardiovascular-related tissues, for example, are of interest to be imaged and/or treated using the emulsions of the invention, including, but limited to, heart tissue and all cardiovascular vessels, angiogenic tissue, any part of a cardiovascular vessel, any material or cell that comes into or caps cardiovascular a vessel, e.g., thrombi, clot or ruptured clot, platelets, muscle cells and the like. Disease conditions to be imaged and/or treated using the emulsions of the invention include, but are not limited to, any disease condition in which vasculature plays an important part in pathology, for example, cardiovascular disease, cancer, areas of inflammation, which may characterize a variety of disorders including rheumatoid arthritis, areas of irritation such as those affected by angioplasty resulting in restenosis, tumors, and areas affected by atherosclerosis. Depending upon the targeting ligand used, emulsions of the invention are of particular use in vascular and/or restenosis imaging. For example, emulsions containing a ligand that bind to αᵥβ₃ integrin are targeted to tissues containing high expression levels of αᵥβ₃ integrin. High expression levels of αᵥβ₃ are typical of activated endothelial cells and are considered diagnostic for neovasculature. Other tissues of interest to be imaged and/or treated include those containing particular malignant tissue and/or tumors.

The combination of target-directed imaging and therapeutic agent delivery allows both the identification of a target and the delivery of the agent in a single procedure, if desired. The ability to image the emulsions delivering the agent provides for identification and/or confirmation of the cells or tissue to which the agent is delivered.

The low and high resolution contrast agents described herein can be used in single-modal or multi-modal imaging. For example, multi-modal imaging can be performed with contrast agents including ancillary reagents that allow for more than one type of imaging such as the combination of X-ray and MRI imaging or other combinations of the types of imaging described herein. Alternatively, more than one contrast agent can be administered to the subject, such that an initial low-resolution imaging technique to localize a low resolution contrast agent is followed by a high resolution imaging technique to localize a higher resolution contrast agent.

In one embodiment, the presence of a target tissue is located using a low-resolution imaging technique. Non-limiting examples of low resolution imaging techniques include X-ray fluoroscopy, MR fluoroscopy, real-time ultrasound, nuclear imaging (e.g., scintigraphy, positron emission tomography (PET), optical imaging (e.g., near-infrared, fluorescent) and single photon emission computed tomography (SPECT)). A higher resolution image is then obtained of the target tissue located using the low resolution imaging technique. As used herein, the term “higher resolution imaging technique” refers to a method that obtains a higher resolution image than the low resolution imaging technique used in the particular embodiment. As used herein, the term “low resolution” indicates that the imagining technique has a higher sensitivity than the higher resolution imaging technique. The higher initial sensitivity allows for a wider field of search to identify potential target tissues, to be followed by higher resolution imaging to obtain more definitive information about the identified target tissue(s). The resolution of the imagining technique is generally determined by calculating time/volume scanned. The low resolution imaging technique used typically requires less time to scan a given volume than the higher resolution imaging technique chosen. Non-limiting examples of higher resolution imaging techniques include proton and fluorine MRI, CT (X-ray CT and electron beam CT), ultrasound, and confocal microscopy. One skilled in the art will readily recognize that the resolutions chosen for the low and higher resolution imaging techniques will depend at least upon the technology used, the contrast agent, the subject anatomy, and the tissue being imaged.

In a further embodiment of the invention, low resolution imaging is used to localize an accumulation of a low resolution contrast agent in one or more tissues or areas of interest, and a higher resolution imaging technique is then used in that localized area to detect an accumulation of a higher resolution contrast agent that is analogously targeted as the low resolution contrast agent. Thus, the use and detection of the low resolution contrast agent serves as a guide in obtaining a higher resolution image of a target tissue.

For use as X-ray contrast agents, the compositions of the present invention generally have a perfluorocarbon concentration of about 10% to about 60% w/v, preferably of about 15% to about 50% w/v, more preferably between about 20% to about 40% w/v. Dosages, administered by intravenous injection, will typically range from about 0.5 mmol/kg to 1.5 mmol/kg, preferably about 0.8 mmol/kg to 1.2 mmol/kg. Imaging is performed using known techniques, preferably X-ray computed tomography.

The ultrasound contrast agents of the present invention are administered, for example, by intravenous injection by infusion at a rate of approximately 3 μL/kg/min. Imaging is performed using known techniques of sonography.

The magnetic resonance imaging contrast agents of the present invention may be used in a similar manner as other MRI agents as described in U.S. Pat. Nos. 5,155,215 and 5,087,440; Margerstadt et al. (1986) Magn. Reson. Med 3:808; Runge et al (1988) Radiology 166:835; and Bousquet et al. (1988) Radiology 166:693. Other agents that may be employed are those set forth in U.S. Pat. No. 6,875,419 which are pH sensitive and can change the contrast properties dependent on pulse. Generally, sterile aqueous solutions of the contrast agents are administered to a patient intravenously in dosages ranging from 0.01 to 1.0 mmoles per kg body weight.

The diagnostic radiopharmaceuticals are administered by intravenous injection, usually in saline solution, at a dose of 1 to 100 mCi per 70 kg body weight, or preferably at a dose of 5 to 50 mCi. Imaging is performed using known procedures.

The therapeutic radiopharmaceuticals are administered, for example, by intravenous injection, usually in saline solution, at a dose of 0.01 to 5 mCi per kg body weight, or preferably at a dose of 0.1 to 4 mCi per kg body weight. For comparable therapeutic radiopharmaceuticals, current clinical practice sets dosage ranges from 0.3 to 0.4 mCi/kg for Zevalin™ to 1-2 mCi/kg for OctreoTher™, a labeled somatostatin peptide. For such therapeutic radiopharmaceuticals, there is a balance between tumor cell kill vs. normal organ toxicity, especially radiation nephritis. At these levels, the balance generally favors the tumor cell effect. These dosages are higher than corresponding imaging isotopes.

As used herein, an “individual” is a vertebrate, preferably a mammal, more preferably a human. Mammals include, but are not limited to, humans, farm animals, sport animals, rodents and pets.

As used herein, an “effective amount” or a “sufficient amount” of a substance is that amount sufficient to effect beneficial or desired results, including clinical results, and, as such, an “effective amount” depends upon the context in which it is being applied. An effective amount can be administered in one or more administrations.

As used herein, the singular form “a”, “an”, and “the” includes plural references unless indicated otherwise. For example, “a” target cell includes one or more target cells.

Any low resolution or high resolution contrast agent can be employed in the methods of the instant invention.

A “contrast agent,” as used herein, refers to a compound employed to improve the visibility of internal body structures in an image, e.g., a CT or MRI scan. The term contrast agent is also referred to herein as an imaging agent. Contrast agents can be administered to the subject by, for example, parenteral injection (e.g., intravenously, intra-arterially, intra-thecally, intra-abdominally, subcutaneously, intramuscularly), orally (e.g., as a tablet or a drink), rectally, or via inhalation.

For example, an X-ray contrast agent can comprise barium sulfate, or can comprise iodine in an organic (non-ionic) compound or in an ionic compound. Examples of organic iodine contrast agents include but are not limited to iohexol, iodixanol, ioversol, iopamidol, and combinations thereof. Examples of ionic contrast agents include but are not limited to iodamide meglumine, iothalamate meglumine, diatrizoate meglumine, amidotrizoate meglumine, diatrizoate sodium, ioxaglate meglumine sodium, iothalamate sodium, iothalamate meglumine sodium, diatrizoate meglumine sodium, and combinations thereof.

In another embodiment, an MRI contrast agent can comprise a paramagnetic contrast agent (such as a gadolinium compound), a superparamagnetic contrast agent (such as iron oxide nanoparticles), a diamagnetic agent (such as barium sulfate), and combinations thereof.

In a further embodiment, a CT contrast agent can comprise iodine (ionic or non-ionic formulations), barium, barium sulfate, Gastrografin (a diatrizoate meglumine and diatrizoate sodium solution), and combinations thereof.

In another embodiment, a PET or SPECT contrast agent can comprise a metal chelate.

The invention contemplates that the contrast agents used herein can be targeted contrast agents. As used herein, the term “targeted” shall mean the use of a target-specific ligand directed to a molecular entity of interest, as described further herein.

In one embodiment of the invention, the low resolution and/or higher resolution contrast agents comprise a perfluorocarbon emulsion. Useful perfluorocarbon emulsions are disclosed in U.S. Pat. Nos. 4,927,623, 5,077,036, 5,114,703, 5,171,755, 5,304,325, 5,350,571, 5,393,524, and 5,403,575 and include those in which the perfluorocarbon compound is perfluorodecalin, perfluorooctane, perfluorodichlorooctane, perfluoro-n-octyl bromide, perfluoroheptane, perfluorodecane, perfluorocyclohexane, perfluoromorpholine, perfluorotripropylamine, perfluortributylamine, perfluorodimethylcyclohexane, perfluorotrimethylcyclohexane, perfluorodicyclohexyl ether, perfluoro-n-butyltetrahydrofuran, and compounds that are structurally similar to these compounds and are partially or fully halogenated (including at least some fluorine substituents) or partially or fully perfluorinated including perfluoroalkylated ether, polyether or crown ether.

Emulsifying agents, for example surfactants, are used to facilitate the formation of emulsions and increase their stability. Typically, aqueous phase surfactants have been used to facilitate the formation of oil-in-water emulsions. A surfactant is any substance that contains both hydrophilic and hydrophobic portions. When added to water or solvents, a surfactant reduces the surface tension.

The lipid/surfactants used to form an outer coating on the nanoparticles (that can contain the coupled ligand or entrap reagents for binding desired components to the surface) include natural or synthetic phospholipids, fatty acids, cholesterols, lysolipids, sphingomyelins, tocopherols, glucolipids, stearylarnines, cardiolipins, plasmalogens, a lipid with ether or ester linked fatty acids, and polymerized lipids. In some instances, the lipid/surfactant can include lipid conjugated polyethylene glycol (PEG). Various commercial anionic, cationic, and nonionic surfactants can also be employed, including Tweens, Spans, Tritons, and the like. In some embodiments, preferred surfactants are phospholipids and cholesterol.

Fluorinated surfactants which are soluble in the oil to be emulsified can also be used. Suitable fluorochemical surfactants include perfluorinated alkanoic acids such as perfluorohexanoic and perfluorooctanoic acids and amidoamine derivatives. These surfactants are generally used in amounts of about 0.01 to 5.0% by weight, and preferably in amounts of about 0.1 to 1.0%. Other suitable fluorochemical surfactants include perfluorinated alcohol phosphate esters and their salts; perfluorinated sulfonamide alcohol phosphate esters and their salts; perfluorinated alkyl sulfonamide; alkylene quaternary ammonium salts; N,N(carboxyl-substituted lower alkyl) perfluorinated alkyl sulfonamides; and mixtures thereof. As used herein, the term “perfluorinated” means that the surfactant contains at least one perfluorinated alkyl group.

Suitable perfluorinated alcohol phosphate esters include the free acids of the diethanolamine salts of mono- and bis(1H, 1H, 2H, 2H-perfluoroalkyl)phosphates. The phosphate salts, available under the tradename ZONYL RP (Dupont, Wilmington, Del.), are converted to the corresponding free acids by known methods. Suitable perfluorinated sulfonamide alcohol phosphate esters are described in U.S. Pat. No. 3,094,547. Suitable perfluorinated sulfonamide alcohol phosphate esters and salts of these include perfluoro-n-octyl-N-ethylsulfonamidoethyl phosphate, bis(perfluoro-n-octyl-N-ethylsulfonamidoethyl) phosphate, the ammonium salt of bis(perfluoro-n-octyl-N-ethylsulfonamidoethyl)phosphate, bis(perfluorodecyl-N-ethylsulfonamidoethyl)-phosphate and bis(perfluorohexyl-N ethylsulfonamidoethyl)phosphate. The preferred formulations use phosphatidylcholine, derivatized-phosphatidylethanolamine and cholesterol as the lipid surfactant.

Other known surfactant additives such as PLURONIC F-68, HAMPOSYL L30 (W. R. Grace Co., Nashua, N.H.), sodium dodecyl sulfate, Aerosol 413 (American Cyanamid Co., Wayne, N.J.), Aerosol 200 (American Cyanamid Co.), LIPOPROTEOL LCO (Rhodia Inc., Mammoth, N.J.), STANDAPOL SH 135 (Henkel Corp., Teaneck, N.J.), FIZUL 10-127 (Finetex Inc., Elmwood Park, N.J.), and CYCLOPOL SBFA 30 (Cyclo Chemicals Corp., Miami, Fla.); amphoterics, such as those sold with the trade names: Deriphat™ 170 (Henkel Corp.), LONZAINE JS (Lonza, Inc.), NIRNOL C2N-SF (Miranol Chemical Co., Inc., Dayton, N.J.), AMPHOTERGE W2 (Lonza, Inc.), and AMPHOTERGE 2WAS (Lonza, Inc.); non-ionics, such as those sold with the trade names: PLURONIC F-68 (BASF Wyandotte, Wyandotte, Mich.), PLURONIC F-127 (BASF Wyandotte), BRIJ 35 (ICI Americas; Wilmington, Del.), TRITON X-100 (Rohm and Haas Co., Philadelphia, Pa.), BRIJ 52 (ICI Americas), SPAN 20 (ICI Americas), GENEROL 122 ES (Henkel Corp.), TRITON N-42 (Rohm and Haas Co.), Triton™ N-101 (Rohm and Haas Co.), TRITON X-405 (Rohm and Haas Co.), TWEEN 80 (ICI Americas), TWEEN 85 (ICI Americas), and BRIJ 56 (ICI Americas) and the like, may be used alone or in combination in amounts of 0.10 to 5.0% by weight to assist in stabilizing the emulsions.

Lipid encapsulated emulsions may be formulated with cationic lipids in the surfactant layer that facilitate entrapping or adhering ligands, such as nucleic acids and aptamers, to particle surfaces. Typical cationic lipids may include DOTMA, N-[1-(2,3-dioleoyloxy)propyl]-N,N,N-trimethylammonium chloride; DOTAP, 1,2-dioleoyloxy-3-(trimethylammonio)propane; DOTB, 1,2-dioleoyl-3-(4′-trimethyl-ammonio)butanoyl-sn-glycerol,1,2-diacyl-3-trimethylammonium-propane; DAP, 1,2-diacyl-3-dimethylammonium-propane; TAP, 1,2-diacyl-3-trimethylammonium-propane; 1,2-diacyl-sn-glycerol-3-ethyl phosphocholine; 3β-[N′, N′-dimethylaminoethane)-carbamol]cholesterol-HCl, DC-Cholesterol (DC-Chol); and DDAB, dimethyldioctadecylammonium bromide. In general the molar ratio of cationic lipid to non-cationic lipid in the lipid surfactant monolayer may be, for example, 1:1000 to 2:1, preferably, between 2:1 to 1:10, more preferably in the range between 1:1 to 1:2.5 and most preferably 1:1 (ratio of mole amount cationic lipid to mole amount non-cationic lipid, e.g., DPPC). A wide variety of lipids may comprise the non-cationic lipid component of the emulsion surfactant, particularly dipalmitoylphosphatidylcholine, dipalmitoylphosphatidyl-ethanolamine or dioleoylphosphatidylethanolamine in addition to those previously described. In lieu of cationic lipids as described above, lipids bearing cationic polymers such as polylysine or polyarginine may also be included in the lipid surfactant and afford binding of a negatively charged therapeutic, such as genetic material or analogues there of, to the outside of the emulsion particles. In some embodiments, the lipids can be cross-linked to provide stability to the emulsions for use in vivo. Emulsions with cross-linked lipids can be particularly useful for imaging methods described herein.

In particular embodiments, included in the lipid/surfactant coating are components with reactive groups that can be used to couple a target-specific ligand and/or the ancillary substance useful for imaging or therapy. In some embodiments, a lipid/surfactant coating which provides a vehicle for binding a multiplicity of copies of one or more desired components to the nanoparticle is preferred. As will be described below, the lipid/surfactant components can be coupled to these reactive groups through functionalities contained in the lipid/surfactant component. For example, phosphatidylethanolamine may be coupled through its amino group directly to a desired moiety, or may be coupled to a linker such as a short peptide which may provide carboxyl, amino, or sulfhydryl groups as described below. Alternatively, standard linking agents such a maleimides may be used. A variety of methods may be used to associate the targeting ligand and the ancillary substances to the nanoparticles; these strategies may include the use of spacer groups such as polyethyleneglycol or peptides, for example.

The lipid/surfactant coated nanoparticles are typically formed by microfluidizing a mixture of the oil which forms the core and the lipid/surfactant mixture which forms the outer layer in suspension in aqueous medium to form an emulsion. In this procedure, the lipid/surfactants may already be coupled to additional ligands when they are emulsified into the nanoparticles, or may simply contain reactive groups for subsequent coupling. Alternatively, the components to be included in the lipid/surfactant layer may simply be solubilized in the layer by virtue of the solubility characteristics of the ancillary material. Sonication or other techniques may be required to obtain a suspension of the lipid/surfactant in the aqueous medium. Typically, at least one of the materials in the lipid/surfactant outer layer comprises a linker or functional group which is useful to bind the additional desired component or the component may already be coupled to the material at the time the emulsion is prepared.

For coupling by covalently binding the target-specific ligand or other organic moiety (such as a chelating agent for a paramagnetic metal) to the components of the outer layer, various types of bonds and linking agents may be employed. Typical methods for forming such coupling include formation of amides with the use of carbodiamides, or formation of sulfide linkages through the use of unsaturated components such as maleimide. Other coupling agents include, for example, glutaraldehyde, propanedial or butanedial, 2-iminothiolane hydrochloride, bifunctional N-hydroxysuccinimide esters such as disuccinimidyl suberate, disuccinimidyl tartrate, bis[2-(succinimidooxycarbonyloxy)ethyl]sulfone, heterobifunctional reagents such as N-(5-azido-2-nitrobenzoyloxy)succinimide, succinimidyl 4-(N-maleimidomethyl)cyclohexane-1-carboxylate, and succinimidyl 4-(p-maleimidophenyl)butyrate, homobifunctional reagents such as 1,5-difluoro-2,4-dinitrobenzene, 4,4′-difluoro-3,3′-dinitrodiphenylsulfone, 4,4′-diisothiocyano-2,2′-disulfonic acid stilbene, p-phenylenediisothiocyanate, carbonylbis(L-methionine p-nitrophenyl ester), 4,4′-dithiobisphenylazide, erythritolbiscarbonate and bifunctional imidoesters such as dimethyl adipimidate hydrochloride, dimethyl suberimidate, dimethyl 3,3′-dithiobispropionimidate hydrochloride and the like. Linkage can also be accomplished by acylation, sulfonation, reductive amination, and the like. A multiplicity of ways to couple, covalently, a desired ligand to one or more components of the outer layer is well known in the art. The ligand itself may be included in the surfactant layer if its properties are suitable. For example, if the ligand contains a highly lipophilic portion, it may itself be embedded in the lipid/surfactant coating. Further, if the ligand is capable of direct adsorption to the coating, this too will affect its coupling. For example, nucleic acids, because of their negative charge, adsorb directly to cationic surfactants.

The ligand may bind directly to the nanoparticle, i.e., the ligand is associated with the nanoparticle itself. Alternatively, indirect binding may also be effected using a hydrolizable anchor, such as a hydrolizable lipid anchor, to couple the targeting ligand or other organic moiety to the lipid/surfactant coating of the emulsion. Indirect binding such as that effected through biotin/avidin may also be employed for the ligand. For example, in biotin/avidin mediated targeting, the targeting ligand is coupled not to the emulsion, but rather coupled, in biotinylated form to the targeted tissue.

Ancillary agents that may be coupled to the contrast agents include radionuclides. Radionuclides may be either therapeutic or diagnostic; diagnostic imaging using such nuclides is well known and by targeting radionuclides to desired tissue a therapeutic benefit may be realized as well. Radionuclides for diagnostic imaging often include gamma emitters (e.g., ⁹⁶Tc) and radionuclides for therapeutic purposes often include alpha emitters (e.g., ²²⁵Ac) and beta emitters (e.g., ⁹⁰Y). Typical diagnostic radionuclides include ^{99m}Tc, ⁹⁶Tc, ⁹⁵Tc, ¹¹¹In, ⁶²Cu, ⁶⁴Cu, ⁶⁷Ga, ⁶⁸Ga, ²⁰¹TI, ⁷⁹Kr, and ¹⁹²Ir, and therapeutic nuclides include ²²⁵Ac, ¹⁸⁶Re, ¹⁸⁸Re, ¹⁵³Sm, ¹⁶⁶Ho, ¹⁷⁷Lu, ¹⁴⁹Pm, ⁹⁰Y, ²¹²Bi, ¹⁰³Pd, ¹⁰⁹Pd, ¹⁵⁹Gd, ¹⁴⁰La, ¹⁹⁸Au, ¹⁹⁹Au, ¹³³Xe, ¹⁶⁹Yb, ¹⁷⁵Yb, ¹⁶⁵Dy, ¹⁶⁶Dy, ¹²³I, ¹³¹I, ⁶⁷Cu, ¹⁰⁵Rh, ¹¹¹Ag, and ¹⁹²Ir. The nuclide can be provided to a preformed emulsion in a variety of ways. For example, ⁹⁹Tc-pertechnate may be mixed with an excess of stannous chloride and incorporated into the preformed emulsion of nanoparticles. Stannous oxinate can be substituted for stannous chloride. In addition, commercially available kits, such as the HM-PAO (exametazine) kit marketed as Ceretek® by Nycomed Amersham can be used. Means to attachvarious radioligands to the contrast agents of the invention are understood in the art.

Chelating agents containing metal ions for use in magnetic resonance imaging can also be employed as ancillary agents. Typically, a chelating agent containing a paramagnetic metal or superparamagnetic metal is associated with the lipids/surfactants of the coating on the nanoparticles and incorporated into the initial mixture which is sonicated. The chelating agent can be coupled directly to one or more of components of the coating layer. Suitable chelating agents are macrocyclic or linear chelating agents and include a variety of multi-dentate compounds including EDTA, DPTA, DOTA, and the like. These chelating agents can be coupled directly to functional groups contained in, for example, phosphatidyl ethanolamine, oleates, or any other synthetic natural or functionalized lipid or lipid soluble compound. Alternatively, these chelating agents can coupled through linking groups.

The paramagnetic and superparamagnetic metals useful in the MRI contrast agents of the invention include rare earth metals, typically, manganese, ytterbium, terbium, gadolinium, europium, and the like. Iron ions may also be used.

A particularly preferred set of MRI chelating agents includes 1,4,7,10-tetraazacyclododecane-1,4,7,10-tetraacetic acid (DOTA) and its derivatives, in particular, a methoxybenzyl derivative (MEO-DOTA) and a methoxybenzyl derivative comprising an isothiocyanate functional group (MEO-DOTA-NCS) which can then be coupled to the amino group of phosphatidyl ethanolamine or to a peptide derivatized form thereof. Derivatives of this type are described in U.S. Pat. No. 5,573,752 and other suitable chelating agents are disclosed in U.S. Pat. No. 6,056,939.

The DOTA isocyanate derivative can also be coupled to the lipid/surfactant directly or through a peptide spacer, such as a gly-gly-gly spacer. For direct coupling, the MEO-DOTA-NCS is simply reacted with phosphoethanolamine (PE) to obtain the coupled product. When a peptide is employed, for example a triglycyl link, PE is first coupled to t-boc protected triglycine. Standard coupling techniques, such as forming the activated ester of the free acid of the t-boc-triglycine using diisopropyl carbodiimide (or an equivalent thereof) with either N-hydroxy succinimide (NHS) or hydroxybenzotriazole (HBT) are employed and the t-boc-triglycine-PE is purified.

Other ancillary agents include fluorophores (such as fluorescein, dansyl, quantum dots, and the like) and infrared dyes or metals may be used in optical or light imaging (e.g., confocal microscopy and fluorescence imaging). For nuclear imaging, such as PET imaging, tosylated and ¹⁸F fluorinated compounds may be associated with the nanoparticles as ancillary agents.

In some embodiments, the biologically active agents are incorporated within the core of the emulsion nanoparticles.

Included in the surface of the nanoparticle, in some embodiments of the invention, are biologically active agents. These biologically active agents can be of a wide variety, including proteins, nucleic acids, pharmaceuticals, and the like. Thus, included among suitable pharmaceuticals are antineoplastic agents, hormones, analgesics, anesthetics, neuromuscular blockers, antimicrobials or antiparasitic agents, antiviral agents, interferons, antidiabetics, antihistamines, antitussives, anticoagulants, and the like.

The targeted emulsions of the invention may also be used to provide a therapeutic agent combined with an imaging agent. Such emulsions would permit, for example, the site to be imaged in order to monitor the progress of the therapy on the site and to make desired adjustments in the dosage or therapeutic agent subsequently directed to the site. The invention thus provides a noninvasive means for the detection and therapeutic treatment of thrombi, infections, cancers and infarctions, for example, in patients while employing conventional imaging systems.

In all of the foregoing cases, whether the associated moiety is a targeting ligand or is an ancillary agent, the defined moiety may be non-covalently associated with the lipid/surfactant layer, may be directly coupled to the components of the lipid/surfactant layer, or may be indirectly coupled to said components through spacer moieties.

The imaging and/or therapeutic target may be an in vivo or in vitro target and, preferably, a biological material although the target need not be a biological material. The target may be comprised of a surface to which the contrast substance binds or a three dimensional structure in which the contrast substance penetrates and binds to portions of the target below the surface.

Preferably, a ligand is incorporated into the contrast emulsion to immobilize or prolong the half-life of the emulsion nanoparticles at the imaging and/or therapeutic target. The ligand may be specific for a desired target to allow active targeting. Active targeting refers to ligand-directed, site-specific accumulation of agents to cells, tissues or organs by localization and binding to molecular epitopes, i.e., receptors, lipids, peptides, cell adhesion molecules, polysaccharides, biopolymers, and the like, presented on the surface membranes of cells or within the extracellular or intracellular matrix. A wide variety of ligands can be used including an antibody, a fragment of an antibody, a polypeptide such as small oligopeptide, a large polypeptide or a protein having three dimensional structure, a peptidomimetic, a polysaccharide, an aptamer, a lipid, a nucleic acid, a lectin or a combination thereof. Generally, the ligand specifically binds to a cellular epitope or receptor.

The term “ligand” as used herein is intended to refer to a targeting molecule that binds specifically to another molecule of a biological target separate and distinct from the emulsion particle itself. The reaction does not require nor exclude a molecule that donates or accepts a pair of electrons to form a coordinate covalent bond with a metal atom of a coordination complex. Thus a ligand may be attached covalently for direct-conjugation or noncovalently for indirect conjugation to the surface of the nanoparticle surface.

In some embodiments, for example for use in vivo, the binding affinity of the ligand for its specific target is about 10⁻⁷ M or greater. In some embodiments, for example, for use in vitro, the binding affinity of the ligand for its specific target can be less than 10⁷ M.

Avidin-biotin interactions are extremely useful, noncovalent targeting systems that have been incorporated into many biological and analytical systems and selected in vivo applications. Avidin has a high affinity for biotin (10⁻¹⁵ M) facilitating rapid and stable binding under physiological conditions. Some targeted systems utilizing this approach are administered in two or three steps, depending on the formulation. Typically in these systems, a biotinylated ligand, such as a monoclonal antibody, is administered first and “pretargeted” to the unique molecular epitopes. Next, avidin is administered, which binds to the biotin moiety of the “pretargeted” ligand. Finally, the biotinylated emulsion is added and binds to the unoccupied biotin-binding sites remaining on the avidin thereby completing the ligand-avidin-emulsion “sandwich.” The avidin-biotin approach can avoid accelerated, premature clearance of targeted agents by the reticuloendothelial system secondary to the presence of surface antibody. Additionally, avidin, with four, independent biotin binding sites provides signal amplification and improves detection sensitivity.

As used herein, the term “biotin emulsion” or “biotinylated” with respect to conjugation to a biotin emulsion or biotin agent is intended to include biotin, biocytin and other biotin derivatives and analogs such as biotin amido caproate N-hydroxysuccinimide ester, biotin 4-amidobenzoic acid, biotinamide caproyl hydrazide and other biotin derivatives and conjugates. Other derivatives include biotin-dextran, biotin-disulfide N-hydroxysuccinimide ester, biotin-6 amido quinoline, biotin hydrazide, d-biotin-N hydroxysuccinimide ester, biotin maleimide, d-biotinp-nitrophenyl ester, biotinylated nucleotides and biotinylated amino acids such as N, epsilon-biotinyl-1-lysine. The term “avidin emulsion” or “avidinized” with respect to conjugation to an avidin emulsion or avidin agent is intended to include avidin, streptavidin and other avidin analogs such as streptavidin or avidin conjugates, highly purified and fractionated species of avidin or streptavidin, and non-amino acid or partial-amino acid variants, recombinant or chemically synthesized avidin.

Targeting ligands may be chemically attached to the surface of nanoparticles of the emulsion by a variety of methods depending upon the nature of the particle surface. Conjugations may be performed before or after the emulsion particle is created depending upon the ligand employed. Direct chemical conjugation of ligands to proteinaceous agents often take advantage of numerous amino-groups (e.g., lysine) inherently present within the surface. Alternatively, functionally active chemical groups such as pyridyldithiopropionate, maleimide or aldehyde may be incorporated into the surface as chemical “hooks” for ligand conjugation after the particles are formed. Another common post-processing approach is to activate surface carboxylates with carbodiimide prior to ligand addition. The selected covalent linking strategy is primarily determined by the chemical nature of the ligand. Antibodies and other large proteins may denature under harsh processing conditions; whereas, the bioactivity of carbohydrates, short peptides, aptamers, drugs or peptidomimetics often can be preserved. To ensure high ligand binding integrity and maximize targeted particle avidity flexible polymer spacer arms, e.g., polyethylene glycol or simple caproate bridges, can be inserted between an activated surface functional group and the targeting ligand. These extensions can be 10 nm or longer and minimize interference of ligand binding by particle surface interactions.

Antibodies, particularly monoclonal antibodies, may also be used as site-targeting ligands directed to any of a wide spectrum of molecular epitopes including pathologic molecular epitopes. Immunoglobin-γ (IgG) class monoclonal antibodies have been conjugated to liposomes, emulsions and other microbubble particles to provide active, site-specific targeting. Generally, these proteins are symmetric glycoproteins (MW ca. 150,000 Daltons) composed of identical pairs of heavy and light chains. Hypervariable regions at the end of each of two arms provide identical antigen-binding domains. A variably sized branched carbohydrate domain is attached to complement-activating regions, and the hinge area contains particularly accessible interchain disulfide bonds that may be reduced to produce smaller fragments.

Preferably, monoclonal antibodies are used in the antibody compositions of the invention. Monoclonal antibodies specific for selected antigens on the surface of cells may be readily generated using conventional techniques (see, for example, U.S. Pat. Nos. RE 32,011, 4,902,614, 4,543,439, and 4,411,993). Hybridoma cells can be screened immunochemically for production of antibodies specifically reactive with an antigen, and monoclonal antibodies can be isolated. Other techniques may also be utilized to construct monoclonal antibodies (see, for example, Huse et al. (1989) Science 246:1275-1281; Sastry et al. (1989) Proc. Natl. Acad Sci. USA 86:5728-5732; Alting-Mees et al. (1990) Strategies in Molecular Biology 3:1-9).

Within the context of the present invention, antibodies are understood to include various kinds of antibodies, including, but not necessarily limited to, naturally occurring antibodies, monoclonal antibodies, polyclonal antibodies, antibody fragments that retain antigen binding specificity (e.g., Fab, and F(ab′)₂) and recombinantly produced binding partners, single domain antibodies, hybrid antibodies, chimeric antibodies, single-chain antibodies, human antibodies, humanized antibodies, and the like. Generally, antibodies are understood to be reactive against a selected antigen of a cell if they bind with an affinity (association constant) of greater than or equal to 10⁷ M⁻¹. Antibodies against selected antigens for use with the emulsions may be obtained from commercial sources.

Further description of the various kinds of antibodies of use as site-targeting ligands in the invention is provided herein, in particular, later in this section.

The emulsions of the present invention also employ targeting agents that are ligands other than an antibody or fragment thereof. For example, polypeptides, like antibodies, may have high specificity and epitope affinity for use as vector molecules for targeted contrast agents. These may be small oligopeptides, having, for example, 5 to 20 amino acids, specific for a unique receptor sequences (such as, for example, the RGD epitope of the platelet GIIbIIIa receptor) or larger, biologically active hormones such as cholecystokinin. Smaller peptides potentially have less inherent immunogenicity than nonhumanized murine antibodies. Peptides or peptide (nonpeptide) analogues of cell adhesion molecules, cytokines, selectins, cadhedrins, Ig superfamily, integrins and the like may be utilized for targeted imaging and/or therapeutic delivery.

In some instances, the ligand is a non-peptide organic molecule, such as those described in U.S. Pat. No. 6,130,231 (for example as set forth in formula 1); U.S. Pat. Nos. 6,153,628; 6,322,770; and PCT publication WO 01/97848. “Non-peptide” moieties in general are those other than compounds which are simply polymers of amino acids, either gene encoded or non-gene encoded. Thus, “non-peptide ligands” are moieties which are commonly referred to as “small molecules” lacking in polymeric character and characterized by the requirement for a core structure other than a polymer of amino acids. The non-peptide ligands useful in the invention may be coupled to peptides or may include peptides coupled to portions of the ligand which are responsible for affinity to the target site, but it is the non-peptide regions of this ligand which account for its binding ability. For example, non-peptide ligands specific for the αᵥβ₃ integrin are described in U.S. Pat. Nos. 6,130,231 and 6,153,628.

Carbohydrate-bearing lipids may be used for targeting of the emulsions, as described, for example, in U.S. Pat. No. 4,310,505.

Asialoglycoproteins have been used for liver-specific applications due to their high affinity for asialoglycoproteins receptors located uniquely on hepatocytes. Asialoglycoproteins directed agents (primarily magnetic resonance agents conjugated to iron oxides) have been used to detect primary and secondary hepatic tumors as well as benign, diffuse liver disease such as hepatitis. The asialoglycoproteins receptor is highly abundant on hepatocytes, approximately 500,000 per cell, rapidly internalizes and is subsequently recycled to the cell surface. Polysaccharides such as arabinogalactan may also be utilized to localize emulsions to hepatic targets. Arabinogalactan has multiple terminal arabinose groups that display high affinity for asialoglycoproteins hepatic receptors.

Aptamers are high affinity, high specificity RNA or DNA-based ligands produced by in vitro selection experiments (SELEX: systematic evolution of ligands by exponential enrichment). Aptamers are generated from random sequences of 20 to 30 nucleotides, selectively screened by absorption to molecular antigens or cells, and enriched to purify specific high affinity binding ligands. To enhance in vivo stability and utility, aptamers are generally chemically modified to impair nuclease digestion and to facilitate conjugation with drugs, labels or particles. Other, simpler chemical bridges often substitute nucleic acids not specifically involved in the ligand interaction. In solution aptamers are unstructured but can fold and enwrap target epitopes providing specific recognition. The unique folding of the nucleic acids around the epitope affords discriminatory intermolecular contacts through hydrogen bonding, electrostatic interaction, stacking, and shape complementarity. In comparison with protein-based ligands, generally aptamers are stable, are more conducive to heat sterilization, and have lower immunogenicity. Aptamers are currently used to target a number of clinically relevant pathologies including angiogenesis, activated platelets, and solid tumors and their use is increasing. The clinical effectiveness of aptamers as targeting ligands for imaging and/or therapeutic emulsion particles may be dependent upon the impact of the negative surface charge imparted by nucleic acid phosphate groups on clearance rates. Previous research with lipid-based particles suggest that negative zeta potentials markedly decrease liposome circulatory half-life, whereas, neutral or cationic particles have similar, longer systemic persistence.

It is also possible to use what has been referred to as a “primer material” to couple specific binding species to the emulsion for certain applications. As used herein, “primer material” refers to any constituent or derivatized constituent incorporated into the emulsion lipid surfactant layer that could be chemically utilized to form a covalent bond between the particle and a targeting ligand or a component of the targeting ligand such as a subunit thereof.

Thus, the specific binding species (i.e., targeting ligand) may be immobilized on the encapsulating lipid monolayer by direct adsorption to the oil/aqueous interface or using a primer material. A primer material may be any surfactant compatible compound incorporated in the particle to chemically couple with or adsorb a specific binding or targeting species. The preferred result is achieved by forming an emulsion with an aqueous continuous phase and a biologically active ligand adsorbed or conjugated to the primer material at the interface of the continuous and discontinuous phases. Naturally occurring or synthetic polymers with amine, carboxyl, mercapto, or other functional groups capable of specific reaction with coupling agents and highly charged polymers may be utilized in the coupling process. The specific binding species (e.g., antibody) may be immobilized on the oil coupled to a high Z number atom emulsion particle surface by direct adsorption or by chemical coupling. Examples of specific binding species which can be immobilized by direct adsorption include small peptides, peptidomimetics, or polysaccharide-based agents. To make such an emulsion the specific binding species may be suspended or dissolved in the aqueous phase prior to formation of the emulsion. Alternatively, the specific binding species may be added after formation of the emulsion and incubated with gentle agitation at room temperature (about 25° C.) in a pH 7.0 buffer (typically phosphate buffered saline) for 1.2 to 18 hours.

Where the specific binding species is to be coupled to a primer material, conventional coupling techniques may be used. The specific binding species may be covalently bonded to primer material with coupling agents using methods which are known in the art. Primer materials may include phosphatidylethanolamine (PE), N-caproylamine-PE, n-dodecanylamine, phosphatidylthioethanol,N-1,2-diacyl-sn-glycero-3-phosphoethanolamine-N-[4-(p-maleimidophenyl)butyramide], 1,2-diacyl-sn-glycero-3-phosphoethanolamine-N-[4-(p-maleimidomethyl)cyclohexane-carboxylate], 1,2-diacyl-sn-glycero-3-phosphoethanolamine-N-[3-(2-pyridyldithio)propionate], 1,2-diacyl-sn-glycero-3-phosphoethanolamine-N[PDP(polyethylene glycol)2000], N-succinyl-PE, N-glutaryl-PE, N-dodecanyl-PE, N-biotinyl-PE, or N-caproyl-PE. Additional coupling agents include, for example, use a carbodiimide or an aldehyde having either ethylenic unsaturation or having a plurality of aldehyde groups. Further description of additional coupling agents appropriate for use is provided herein, in particular, later in this section.

Covalent bonding of a specific binding species to the primer material can be carried out with the reagents provided herein by conventional, well-known reactions, for example, in the aqueous solutions at a neutral pH, at temperatures of less than 25° C. for 1 hour to overnight. Examples of linkers for coupling a ligand, including non-peptide ligands, are known in the art.

Emulsifying and/or solubilizing agents may also be used in conjunction with emulsions. Such agents include, but are not limited to, acacia, cholesterol, diethanolamine, glyceryl monostearate, lanolin alcohols, lecithin, mono- and di-glycerides, mono-ethanolamine, oleic acid, oleyl alcohol, poloxamer, peanut oil, palmitic acid, polyoxyethylene 50 stearate, polyoxyl 35 castor oil, polyoxyl 10 oleyl ether, polyoxyl 20 cetostearyl ether, polyoxyl 40 stearate, polysorbate 20, polysorbate 40, polysorbate 60, polysorbate 80, propylene glycol diacetate, propylene glycol monostearate, sodium lauryl sulfate, sodium stearate, sorbitan mono-laurate, sorbitan mono-oleate, sorbitan mono-palmitate, sorbitan monostearate, stearic acid, trolamine, and emulsifying wax. All lipids with perfluoro fatty acids as a component of the lipid in lieu of the saturated or unsaturated hydrocarbon fatty acids found in lipids of plant or animal origin may be used. Suspending and/or viscosity-increasing agents that may be used with emulsions include, but are not limited to, acacia, agar, alginic acid, aluminum mono-stearate, bentonite, magma, carbomer 934P, carboxymethylcellulose, calcium and sodium and sodium 12, carrageenan, cellulose, dextrin, gelatin, guar gum, hydroxyethyl cellulose, hydroxypropyl methylcellulose, magnesium aluminum silicate, methylcellulose, pectin, polyethylene oxide, polyvinyl alcohol, povidone, propylene glycol alginate, silicon dioxide, sodium alginate, tragacanth, and xanthum gum.

As described herein, emulsions of the invention may incorporate bioactive agents (e.g., drugs, prodrugs, genetic materials, radioactive isotopes, or combinations thereof) in their native form or derivatized with hydrophobic or charged moieties to enhance incorporation or adsorption to the nanoparticle. In particular, bioactive agents may be incorporated in targeted emulsions of the invention. The bioactive agent may be a prodrug, including the prodrugs described, for example, by Sinkyla et al. (1975) J. Pharm. Sci. 64:181-210, Koning et al. (1999) Br. J. Cancer 80:1718-1725, U.S. Pat. No. 6,090,800 and U.S. Pat. No. 6,028,066.

Such therapeutic emulsions may also include, but are not limited to antineoplastic agents, radiopharmaceuticals, protein and nonprotein natural products or analogues/mimetics thereof including hormones, analgesics, muscle relaxants, narcotic agonists, narcotic agonist-antagonists, narcotic antagonists, nonsteroidal anti-inflammatories, anesthetic and sedatives, neuromuscular blockers, antimicrobials, anti-helmintics, antimalarials, antiparasitic agents, antiviral agents, antiherpetic agents, antihypertensives, antidiabetic agents, gout related medicants, antihistamines, antiulcer medicants, anticoagulants and blood products.

Genetic material, includes, for example, nucleic acids, RNA and DNA, of either natural or synthetic origin, including recombinant RNA and DNA and antisense RNA and DNA; hammerhead RNA, ribozymes, hammerhead ribozymes, antigene nucleic acids, both single and double stranded RNA and DNA and analogs thereof, immunostimulatory nucleic acid, ribooligonucleotides, antisense ribooligonucleotides, deoxyribooligonucleotides, and antisense deoxyribooligonucleotides. Other types of genetic material that may be used include, for example, genes carried on expression vectors such as plasmids, phagemids, cosmids, yeast artificial chromosomes, and defective or “helper” viruses, antigene nucleic acids, both single and double stranded RNA and DNA and analogs thereof, such as phosphorothioate and phosphorodithioate oligodeoxynucleotides. Additionally, the genetic material may be combined, for example, with proteins or other polymers.

Further description of additional therapeutic agents appropriate for use is provided herein, in particular, later in this section.

As described herein, the emulsion nanoparticles may incorporate on the particle paramagnetic or super paramagnetic elements including but not limited to gadolinium, magnesium, iron, manganese in their native or in a chemically complexed form. Similarly, radioactive nuclides including positron-emitters, gamma-emitters, beta-emitters, alpha-emitters in their native or chemically-complexed form may be included on or in the particles. Adding of these moieties permits the additional use of multiple clinical imaging modalities.

Photoactive agents, i.e. compounds or materials that are active in light or that respond to light, including, for example, chromophores (e.g., materials that absorb light at a given wavelength), fluorophores (e.g., materials that emit light at a given wavelength), photosensitizers (e.g., materials that can cause necrosis of tissue and/or cell death in vitro and/or in vivo), fluorescent materials, phosphorescent materials and the like, that may be used in diagnostic or therapeutic applications. “Light” refers to all sources of light including the ultraviolet (UV) region, the visible region and/or the infrared (IR) region of the spectrum. Suitable photoactive agents that may be used in the present invention have been described by others (for example, U.S. Pat. No. 6,123,923). Further description of additional photoactive agents appropriate for use is provided herein, in particular, later in this section.

In addition, certain ligands, such as, for example, antibodies, peptide fragments, or mimetics of a biologically active ligand may contribute to the inherent therapeutic effects, either as an antagonistic or agonistic, when bound to specific epitopes. As an example, antibody against αᵥβ₃ integrin on neovascular endothelial cells has been shown to transiently inhibit growth and metastasis of solid tumors. The efficacy of therapeutic emulsion particles directed to the αᵥβ₃ integrin may result from the improved antagonistic action of the targeting ligand in addition to the effect of the therapeutic agents incorporated and delivered by particle itself.

Useful emulsions may have a wide range of nominal particle diameters, e.g., from as small as about 0.01 μm to as large as 10 μm, preferably about 50 nm to about 1000 nm, more preferably about 50 nm to about 500 nm, in some instances about 50 nm to about 300 nm, in some instances about 100 nm to about 300 nm, in some instances about 200 nm to about 250 nm, in some instances about 200 nm, in some instances about less than 200 nm. Generally, smaller sized particles, for example, submicron particles, circulate longer and tend to be more stable than larger particles.

In addition to that described elsewhere herein, following is further description of the various kinds of antibodies appropriate for use as site-targeting ligands in and/or with the emulsions of the invention.

Bivalent F(ab′)₂ and monovalent F(ab) fragments can be used as ligands and these are derived from selective cleavage of the whole antibody by pepsin or papain digestion, respectively. Antibodies can be fragmented using conventional techniques and the fragments (including “Fab” fragments) screened for utility in the same manner as described above for whole antibodies. The “Fab” region refers to those portions of the heavy and light chains which are roughly equivalent, or analogous, to the sequences which comprise the branch portion of the heavy and light chains, and which have been shown to exhibit immunological binding to a specified antigen, but which lack the effector Fc portion. “Fab” includes aggregates of one heavy and one light chain (commonly known as Fab′), as well as tetramers containing the 2H and 2L chains (referred to as F(ab)₂), which are capable of selectively reacting with a designated antigen or antigen family. Methods of producing Fab fragments of antibodies are known within the art and include, for example, proteolysis, and synthesis by recombinant techniques. For example, F(ab′)₂ fragments can be generated by treating antibody with pepsin. The resulting F(ab′)₂ fragment can be treated to reduce disulfide bridges to produce Fab′ fragments. “Fab” antibodies may be divided into subsets analogous to those described herein, i.e., “hybrid Fab”, “chimeric Fab”, and “altered Fab”. Elimination of the Fc region greatly diminishes the immunogenicity of the molecule, diminishes nonspecific liver uptake secondary to bound carbohydrate, and reduces complement activation and resultant antibody-dependent cellular toxicity. Complement fixation and associated cellular cytotoxicity can be detrimental when the targeted site must be preserved or beneficial when recruitment of host killer cells and target-cell destruction is desired (e.g., anti-tumor agents).

Most monoclonal antibodies are of murine origin and are inherently immunogenic to varying extents in other species. Humanization of murine antibodies through genetic engineering has lead to development of chimeric ligands with improved biocompatibility and longer circulatory half-lives. Antibodies used in the invention include those that have been humanized or made more compatible with the individual to which they will be administered. In some cases, the binding affinity of recombinant antibodies to targeted molecular epitopes can be improved with selective site-directed mutagenesis of the binding idiotype. Methods and techniques for such genetic engineering of antibody molecules are known in the art. By “humanized” is meant alteration of the amino acid sequence of an antibody so that fewer antibodies and/or immune responses are elicited against the humanized antibody when it is administered to a human. For the use of the antibody in a mammal other than a human, an antibody may be converted to that species format.

Phage display techniques may be used to produce recombinant human monoclonal antibody fragments against a large range of different antigens without involving antibody-producing animals. In general, cloning creates large genetic libraries of corresponding DNA (cDNA) chains deducted and synthesized by means of the enzyme “reverse transcriptase” from total messenger RNA (mRNA) of human B lymphocytes. By way of example, immunoglobulin cDNA chains are amplified by polymerase chain reaction (PCR) and light and heavy chains specific for a given antigen are introduced into a phagemid vector. Transfection of this phagemid vector into the appropriate bacteria results in the expression of an scFv immunoglobulin molecule on the surface of the bacteriophage. Bacteriophages expressing specific immunoglobulin are selected by repeated immunoadsorption/phage multiplication cycles against desired antigens (e.g., proteins, peptides, nuclear acids, and sugars). Bacteriophages strictly specific to the target antigen are introduced into an appropriate vector, (e.g., Escherichia coli, yeast, cells) and amplified by fermentation to produce large amounts of human antibody fragments, generally with structures very similar to natural antibodies. Phage display techniques are known in the art and have permitted the production of unique ligands for targeting and therapeutic applications.

Polyclonal antibodies against selected antigens may be readily generated by one of ordinary skill in the art from a variety of warm-blooded animals such as horses, cows, various fowl, rabbits, mice, or rats. In some cases, human polyclonal antibodies against selected antigens may be purified from human sources.

As used herein, a “single domain antibody” (dAb) is an antibody which is comprised of a V_{H} domain, which reacts immunologically with a designated antigen. A dAb does not contain a V_{L} domain, but may contain other antigen binding domains known to exist in antibodies, for example, the kappa and lambda domains. Methods for preparing dAbs are known in the art. See, for example, Ward et al. (1989) Nature 341:544-546. Antibodies may also be comprised of V_{H} and V_{L} domains, as well as other known antigen binding domains. Examples of these types of antibodies and methods for their preparation are known in the art (see, e.g., U.S. Pat. No. 4,816,467).

Further exemplary antibodies include “univalent antibodies”, which are aggregates comprised of a heavy chain/light chain dimer bound to the Fc (i.e., constant) region of a second heavy chain. This type of antibody generally escapes antigenic modulation. See, e.g., Glennie et al. (1982) Nature 295:712-714.

“Hybrid antibodies” are antibodies wherein one pair of heavy and light chains is homologous to those in a first antibody, while the other pair of heavy and light chains is homologous to those in a different second antibody. Typically, each of these two pairs will bind different epitopes, particularly on different antigens. This results in the property of “divalence”, i.e., the ability to bind two antigens simultaneously. Such hybrids may also be formed using chimeric chains, as set forth herein.

The invention also encompasses “altered antibodies”, which refers to antibodies in which the naturally occurring amino acid sequence in a vertebrate antibody has been varied. Utilizing recombinant DNA techniques, antibodies can be redesigned to obtain desired characteristics. The possible variations are many, and range from the changing of one or more amino acids to the complete redesign of a region, for example, the constant region. Changes in the variable region may be made to alter antigen binding characteristics. The antibody may also be engineered to aid the specific delivery of an emulsion to a specific cell or tissue site. The desired alterations may be made by known techniques in molecular biology, e.g., recombinant techniques, site directed mutagenesis, and other techniques.

“Chimeric antibodies” are antibodies in which the heavy and/or light chains are fusion proteins. Typically the constant domain of the chains is from one particular species and/or class, and the variable domains are from a different species and/or class. The invention includes chimeric antibody derivatives, i.e., antibody molecules that combine a non-human animal variable region and a human constant region. Chimeric antibody molecules can include, for example, the antigen binding domain from an antibody of a mouse, rat, or other species, with human constant regions. A variety of approaches for making chimeric antibodies have been described and can be used to make chimeric antibodies containing the immunoglobulin variable region which recognizes selected antigens on the surface of targeted cells and/or tissues. See, for example, Morrison et al. (1985) Proc. Natl. Acad. Sci. U.S.A. 81:6851; Takeda et al. (1985) Nature 314:452; U.S. Pat. Nos. 4,816,567 and 4,816,397; European Patent Publications EP171496 and EP173494; United Kingdom patent GB 2177096B.

Bispecific antibodies may contain a variable region of an anti-target site antibody and a variable region specific for at least one antigen on the surface of the lipid-encapsulated emulsion. In other cases, bispecific antibodies may contain a variable region of an anti-target site antibody and a variable region specific for a linker molecule. Bispecific antibodies may be obtained forming hybrid hybridomas, for example by somatic hybridization. Hybrid hybridomas may be prepared using the procedures known in the art such as those disclosed in Staerz et al. (1986, Proc. Natl. Acad. Sci. U.S.A. 83:1453) and Staerz et al. (1986, Immunology Today 7:241). Somatic hybridization includes fusion of two established hybridomas generating a quadroma (Milstein et al. (1983) Nature 305:537-540) or fusion of one established hybridoma with lymphocytes derived from a mouse immunized with a second antigen generating a trioma (Nolan et al. (1990) Biochem. Biophys. Acta 1040:1-11). Hybrid hybridomas are selected by making each hybridoma cell line resistant to a specific drug-resistant marker (De Lau et al. (1989) J. Immunol. Methods 117:1-8), or by labeling each hybridoma with a different fluorochrome and sorting out the heterofluorescent cells (Karawajew et al. (1987) J. Immunol. Methods 96:265-270).

Bispecific antibodies may also be constructed by chemical means using procedures such as those described by Staerz et al. (1985) Nature 314:628 and Perez et al. (1985) Nature 316:354. Chemical conjugation may be based, for example, on the use of homo- and heterobifunctional reagents with E-amino groups or hinge region thiol groups. Homobifunctional reagents such as 5,5′-dithiobis(2-nitrobenzoic acid) (DNTB) generate disulfide bonds between the two Fabs, and 0-phenylenedimaleimide (O-PDM) generate thioether bonds between the two Fabs (Brenner et al. (1985) Cell 40:183-190, Glennie et al. (1987) J. Immunol. 139:2367-2375). Heterobifunctional reagents such as N-succinimidyl-3-(2-pyridylditio)propionate (SPDP) combine exposed amino groups of antibodies and Fab fragments, regardless of class or isotype (Van Dijk et al. (1989) Int. J. Cancer 44:738-743).

Bifunctional antibodies may also be prepared by genetic engineering techniques. Genetic engineering involves the use of recombinant DNA based technology to ligate sequences of DNA encoding specific fragments of antibodies into plasmids, and expressing the recombinant protein. Bispecific antibodies can also be made as a single covalent structure by combining two single chains Fv (scFv) fragments using linkers (Winter et al. (1991) Nature 349:293-299); as leucine zippers coexpressing sequences derived from the transcription factors fos and jun (Kostelny et al. (1992) J. Immunol. 148:1547-1553); as helix-turn-helix coexpressing an interaction domain of p53 (Rheinnecker et al. (1996) J. Immunol. 157:2989-2997), or as diabodies (Holliger et al. (1993) Proc. Natl. Acad. Sci. U.S.A. 90:6444-6448).

In addition to that described elsewhere herein, following is further description of coupling agents appropriate for use in coupling a primer material, for example, to a specific binding or targeting ligand. Additional coupling agents use a carbodiimide such as 1-ethyl-3-(3-N,N dimethylaminopropyl)carbodiimide hydrochloride or 1-cyclohexyl-3-(2-morpholinoethyl)carbodiimide methyl-p-toluenesulfonate. Other suitable coupling agents include aldehyde coupling agents having either ethylenic unsaturation such as acrolein, methacrolein, or 2-butenal, or having a plurality of aldehyde groups such as glutaraldehyde, propanedial or butanedial. Other coupling agents include 2-iminothiolane hydrochloride, bifunctional N-hydroxysuccinimide esters such as disuccinimidyl substrate, disuccinimidyl tartrate, bis[2-(succinimidooxycarbonyloxy)ethyl]sulfone, disuccinimidyl propionate, ethylene glycolbis(succinimidyl succinate); heterobifunctional reagents such as N-(5-azido-2-nitrobenzoyloxy)succinimide, p-azidophenylbromide, p-azidophenylglyoxal, 4-fluoro-3-nitrophenylazide, N-hydroxysuccinimidyl-4-azidobenzoate, m-maleimidobenzoyl N-hydroxysuccinimide ester, methyl-4-azidophenylglyoxal, 4-fluoro-3-nitrophenyl azide,N-hydroxysuccinimidyl-4-azidobenzoate hydrochloride, p-nitrophenyl 2-diazo-3,3,3-trifluoropropionate, N-succinimidyl-6-(4′-azido-2′-nitrophenylamino)hexanoate, succinimidyl 4-(N-maleimidomethyl)cyclohexane-1-carboxylate, succinimidyl 4-(p-maleimidophenyl)butyrate, N-succinimidyl(4-azidophenyldithio)propionate, N-succinimidyl 3-(2-pyridyldithio)propionate, N-(4-azidophenylthio)phthalamide; homobifunctional reagents such as 1,5-difluoro-2,4-dinitrobenzene, 4,4′-difluoro-3,3′-dinitrodiphenylsulfone, 4,4′-diisothiocyano-2,2′-disulfonic acid stilbene, p-phenylenediisothiocyanate, carbonylbis(L-methionine p-nitrophenyl ester), 4,4′-dithiobisphenylazide, erythritolbiscarbonate and bifunctional imidoesters such as dimethyl adipimidate hydrochloride, dimethyl suberimidate, dimethyl 3,3′-dithiobispropionimidate hydrochloride and the like.

In addition to that described elsewhere herein, following is further description of therapeutic agents that may be incorporated onto and/or within the nanoparticles of the invention. Generally, the therapeutic agents can be derivatized with a lipid anchor to make the agent lipid soluble or to increase its solubility in lipid, therefor increasing retention of the agent in the lipid layer of the emulsion and/or in the lipid membrane of the target cell. Such therapeutic emulsions may also include, but are not limited to antineoplastic agents, including platinum compounds (e.g., spiroplatin, cisplatin, and carboplatin), methotrexate, fluorouracil, adriamycin, mitomycin, ansamitocin, bleomycin, cytosine arabinoside, arabinosyl adenine, mercaptopolylysine, vincristine, busulfan, chlorambucil, melphalan (e.g., PAM, L-PAM or phenylalanine mustard), mercaptopurine, mitotane, procarbazine hydrochloride dactinomycin (actinomycin D), daunorubicin hydrochloride, doxorubicin hydrochloride, taxol, plicamycin (mithramycin), aminoglutethimide, estramustine phosphate sodium, flutamide, leuprolide acetate, megestrol acetate, tamoxifen citrate, testolactone, trilostane, amsacrine (m-AMSA), asparaginase (L-asparaginase) Erwina asparaginase, interferon α-2a, interferon α-2b, teniposide (VM-26), vinblastine sulfate (VLB), vincristine sulfate, bleomycin, bleomycin sulfate, methotrexate, adriamycin, arabinosyl, hydroxyurea, procarbazine, dacarbazine, mitotic inhibitors such as etoposide and other vinca alkaloids; radiopharmaceuticals such as but not limited to radioactive iodine, samarium, strontium cobalt, yittrium and the like; protein and nonprotein natural products or analogues/mimetics thereof including hormones such as but not limited to growth hormone, somatostatin, prolactin, thyroid, steroids, androgens, progestins, estrogens and antiestrogens; analgesics including but not limited to antirheumatics, such as auranofin, methotrexate, azathioprine, sulfazalazine, leflunomide, hydrochloroquine, and etanercept; muscle relaxants such as baclofen, dantrolene, carisoprodol, diazepam, metaxalone, cyclobenzaprine, chlorzoxazone, tizanidine; narcotic agonists such as codeine, fentanyl, hydromorphone, lleavorphanol, meperidine, methadone, morphine, oxycodone, oxymorphone, propoxyphene; narcotic agonist-antagonists such as buprenorphine, butorphanol, dezocine, nalbuphine, pentazocine; narcotic antagonists such as nalmefene and naloxone, other analgesics including ASA, acetominophen, tramadol, or combinations thereof; nonsteroidal anti-inflammatories including but not limited to celecoxib, diclofenac, diflunisal, etodolac, fenoprofen, flurbiprofen, ibuprofen, indomethacin, ketoprofen, ketorolac, naproxen, oxaproxen, rofecoxib, salisalate, suldindac, tolmetin; anesthetic and sedatives such as etomidate, fentanyl, ketamine, methohexital, propofol, sufentanil, thiopental, and the like; neuromuscular blockers such as but not limited to pancuronium, atracurium, cisatracurium, rocuronium, succinylcholine, vercuronium; antimicrobials including aminoglycosides, antifungal agents including amphotericin B, clotrimazole, fluconazole, flucytosine, griseofulvin, itraconazole, ketoconazole, nystatin, and terbinafine; anti-helmintics; antimalarials, such as chloroquine, doxycycline, mefloquine, primaquine, quinine; antimycobacterial including dapsone, ethambutol, ethionamide, isoniazid, pyrazinamide, rifabutin, rifampin, rifapentine; antiparasitic agents including albendazole, atovaquone, iodoquinol, ivermectin,mebendazole, metronidazole, pentamidine, praziquantel, pyrantel, pyrimethamine, thiabendazole; antiviral agents including abacavir, didanosine, lamivudine, stavudine, zalcitabine, zidovudine as well as protease inhibitors such as indinavir and related compounds, anti-CMV agents including but not limited to cidofovir, foscarnet, and ganciclovir; antiherpetic agents including amatadine, rimantadine, zanamivir; interferons, ribavirin, rebetron; carbapenems, cephalosporins, fluoroquinones, macrolides, penicillins, sulfonamides, tetracyclines, and other antimicrobials including aztreonam, chloramphenieol, fosfomycin, furazolidone, nalidixic acid, nitrofurantoin, vancomycin and the like; nitrates, antihypertensives including diuretics, beta blockers, calcium channel blockers, angiotensin converting enzyme inhibitors, angiotensin receptor antagonists, antiadrenergic agents, anti-dysrhythmics, antihyperlipidemic agents, antiplatelet compounds, pressors, thrombolytics, acne preparations, antipsoriatics; corticosteroids; androgens, anabolic steroids, bisphosphonates; sulfonoureas and other antidiabetic agents; gout related medicants; antihistamines, antitussive, decongestants, and expectorants; antiulcer medicants including antacids, 5-HT receptor antagonists, H2-antagonists, bismuth compounds, proton pump inhibitors, laxatives, octreotide and its analogues/mimetics; anticoagulants; immunization antigens, immunoglobins, immunosuppressive agents; anticonvulsants, 5-HT receptor agonists, other migraine therapies; parkinsonian agents including anticholinergics, and dopaminergics; estrogens, GnRH agonists, progestins, estrogen receptor modulators, tocolytics, uterotnics, thyroid agents such as iodine products and anti-thyroid agents; blood products such as parenteral iron, hemin, hematoporphyrins and their derivatives.

In addition to that described elsewhere herein, following is further description of additional photoactive agents appropriate for use in optical imaging of the nanoparticles of the invention. Suitable photoactive agents include but are not limited to, for example, fluoresceins, indocyanine green, rhodamine, triphenylmethines, polymethines, cyanines, fullerenes, oxatellurazoles, verdins, rhodins, perphycenes, sapphyrins, rubyrins, cholesteryl 4,4-difluoro-5,7-dimethyl-4-bora-3a,4a-diaza-s-indacene-3-dodecanoate, cholesteryl 12-(N-methyl-N-(7-nitrobenz-2-oxa-1,3-diazol-4-yl)amino)dodecanate, cholesteryl cis-parinarate, cholesteryl 3-((6-phenyl)-1,3,5-hexatrienyl)phenyl-proprionate, cholesteryl 1-pyrenebutyrate, cholesteryl-1-pyrenedecanoate, cholesteryl 1-pyrenehexanoate, 22-(N-(7-nitrobenz-2-oxa-1,3-diazol-4-yl)amino)-23,24-bisnor-5-cholen-3β-ol, 22-(N-(7-nitrobenz-2-oxa-1,3-diazol-4-yl)amino)-23,24-bisnor-5-cholen-3β-yl cis-9-octadecenoate, 1-pyrenemethyl3-hydroxy-22,23-bisnor-5-cholenate, 1-pyrene-methyl 3β-(cis-9-octadecenoyloxy)-22,23-bisnor-5-cholenate, acridine orange 10-dodecyl bromide, acridine orange 10-nonyl bromide, 4-(N,N-dimethyl-N-tetradecylammonium)-methyl-7-hydroxycoumarin) chloride, 5-dodecanoylaminofluorescein, 5-dodecanoylaminofluorescein-bis-4,5-dimethoxy-2-nitrobenzyl ether, 2-dodecylresorufin, fluorescein octadecyl ester, 4-heptadecyl-7-hydroxycoumarin, 5-hexadecanoylaminoeosin, 5-hexadecanoylaminofluorescein, 5-octadecanoylaminofluorescein, N-octadecyl-N′-(5-(fluoresceinyl))thiourea, octadecyl rhodamine B chloride, 2-(3-(diphenylhexatrienyl)-propanoyl)-1-hexadecanoyl-sn-glycero-3-phosphocholine, 6-N-(7-nitrobenz-2-oxa-1,3-diazol-4-yl)amino)hexanoic acid, 1-hexadecanoyl-2-(1-pyrenedecanoyl)-sn-glycero-3-phosphocholine, 1,1′-dioctadecyl-3,3,3′,3′-tetramethyl-indocarbocyanine perchlorate, 12-(9-anthroyloxy)oleic acid, 5-butyl-4,4-difluoro-4-bora-3a,4a-diaza-s-indacene-3-nonanoic acid, N-(Lissamine™ rhodamine B sulfonyl)-1,2-dihexadecanoyl-sn-glycero-3-phosphoethanolamine, triethylammonium salt, phenylglyoxal monohydrate, naphthalene-2,3-dicarboxaldehyde, 8-bromomethyl-4,4-difluoro-1,3,5,7-tetramethyl-4-bora-3a,4a-diaza-s-indacene, o-phthaldialdehyde, Lissamine™ rhodamine B sulfonyl chloride, 2′,7′-difluorofluorescein, 9-anthronitrile, 1-pyrenesulfonyl chloride, 4-(4-(dihexadecylamino)-styryl)-N-methylpyridinium iodide, chlorins, such as chlorin, chlorin e6, bonellin, mono-L-aspartyl chlorin e6, mesochlorin, mesotetraphenylisobacteriochlorin, and mesotetraphenylbacteriochlorin, hypocrellin B, purpurins, such as octaethylpurpurin, zinc(II) etiopurpurin, tin(IV) etiopurpurin and tin ethyl etiopurpurin, lutetium texaphyrin, photofrin, metalloporphyrins, protoporphyrin IX, tin protoporphyrin, benzoporphyrin, haematoporphyrin, phthalocyanines, naphthocyanines, merocyanines, lanthanide complexes, silicon phthalocyanine, zinc phthalocyanine, aluminum phthalocyanine, Ge octabutyoxyphthalocyanines, methyl pheophorbide-α-(hexyl-ether), porphycenes, ketochlorins, sulfonated tetraphenylporphines, δ-aminolevulinic acid, texaphyrins, including, for example, 1,2-dinitro-4-hydroxy-5-methoxybenzene, 1,2-dinitro-4-(1-hydroxyhexyl)oxy-5-methoxybenzene, 4-(1-hydroxyhexyl)oxy-5-methoxy-1,2-phenylenediamine, and texaphyrin-metal chelates, including the metals Y(III), Mn(II), Mn(III), Fe(II), Fe(III) and the lanthanide metals Gd(III), Dy(III), Eu(III), La(III), Lu(III) and Tb(III), chlorophyll, carotenoids, flavonoids, bilins, phytochromes, phycobilins, phycoerythrins, phycocyanines, retinoic acids, retinoins, retinates, or combinations of any of the above.

One skilled in the art will readily recognize or can readily determine which of the above compounds are, for example, fluorescent materials and/or photosensitizers. LISSAMINE is the trademark for N-ethyl-N-[4-[[4-[ethyl [(3-sulfophenyl)methyl]amino]phenyl](4-sulfopheny-1)-methylene]-2,5-cyclohexadien-1-ylidene]-3-sulfobenzene-methanaminium hydroxide, inner salt, disodium salt and/or ethyl[4[p[ethyl(m-sulfobenzyl)amino]-α-(p-sulfophenyl)benzylidene]-2,5-cyclohexadien-1-ylidene](m-sulfobenzyl)ammonium hydroxide inner salt disodium salt (commercially available from Molecular Probes, Inc., Eugene, Oreg.). Other suitable photoactive agents for use in the present invention include those described in U.S. Pat. No. 4,935,498, such as a dysprosium complex of 4,5,9,24-tetraethyl-16-(1-hydroxyhexyl)oxy-17 methoxypentaazapentacyclo-(20.2.1.1³,6.1⁸,11.0¹⁴,19)-heptacosa-1,3,5,7,9,11(27),12,14,16,18,20,22(25),23-tridecaene and dysprosium complex of 2-cyanoethyl-N,N-diisopropyl-6-(4,5,9,24-tetraethyl-17-methoxypentaazapent acyclo-(20.2.1.1³,6.1⁸,11.0¹⁴,19)-heptacosa-1,3,5,7,9,11(27), 12,14,16,18,20,22(25),23-tridecaene-16-(1-oxy)hexylphosphoramidite.

Methods of Preparation of the Compositions

The emulsions of the present invention may be prepared by various techniques, discussed in detail in PCT application PCT/US2004/025484. In a typical procedure for preparing the emulsions of the invention, a perfluorocarbon and the components of the lipid/surfactant coating are fluidized in aqueous medium to form an emulsion. The functional components of the surface layer may be included in the original emulsion, or may later be covalently coupled to the surface layer subsequent to the formation of the nanoparticle emulsion. In one particular instance, for example, where a nucleic acid targeting agent or drug is to be included, the coating may employ a cationic surfactant and the nucleic acid adsorbed to the surface after the particle is formed.

Generally, the emulsifying process involves directing high pressure streams of mixtures containing the aqueous solution, a primer material or the specific binding species, a perfluorocarbon and a surfactant (if any) so that they impact one another to produce emulsions of narrow particle size and distribution. The MICROFLUIDIZER apparatus (Microfluidics, Newton, Mass.) can be used to make the preferred emulsions. The apparatus is also useful to post-process emulsions made by sonication or other conventional methods. Feeding a stream of emulsion droplets through the MICROFLUIDIZER apparatus yields formulations small size and narrow particle size distribution.

An alternative method for making the emulsions involves sonication of a mixture of a perfluorocarbon and an aqueous solution containing a suitable primer material and/or specific binding species. Generally, these mixtures include a surfactant. Cooling the mixture being emulsified, minimizing the concentration of surfactant, and buffering with a saline buffer will typically maximize both retention of specific binding properties and the coupling capacity of the primer material. These techniques provide excellent emulsions with high activity per unit of absorbed primer material or specific binding species.

When high concentrations of a primer material or specific binding species are coated on lipid emulsions, the mixture should be heated during sonication and have a relatively low ionic strength and moderate to low pH. Too low an ionic strength, too low a pH or too much heat may cause some degradation or loss of all of the useful binding properties of the specific binding species or the coupling capacity of the primer material. Careful control and variation of the emulsification conditions can optimize the properties of the primer material or the specific binding species while obtaining high concentrations of coating. Prior to administration, these formations may be rendered sterile with techniques known in the art, for example, terminal steam sterilization.

The emulsion particle sizes can be controlled and varied by modification of the emulsification techniques and the chemical components. Techniques and equipment for determining particle sizes are known in the art and include, but not limited to, laser light scattering and an analyzer for determining laser light scattering by particles.

When appropriately prepared, the nanoparticles that comprise ancillary agents contain a multiplicity of functional such agents at their outer surface, the nanoparticles typically contain tens, hundreds or thousands of molecules of the biologically active agent, targeting ligand, radionuclide, MRI contrast agent and/or PET contrast agent. For MRI contrast agents, the number of copies of a component to be coupled to the nanoparticle is typically in excess of about 5,000 copies per particle, more preferably in excess of about 10,000 copies per particle, still more preferably in excess of about 30,000 copies per particle, and still more preferably about 50,000-100,000 or more copies per particle. The number of targeting agents per particle is typically less, of the order of several hundred while the concentration of PET contrast agents, fluorophores, radionuclides, and biologically active agents is also variable.

The nanoparticles need not contain an ancillary agent. In general, because the particles have a perfluorocarbon core, X-ray imaging and, in some cases, ultrasound imaging can be used to track the location of the particles concomitantly with any additional functions described herein. Additionally, such particles coupled to a targeting ligand are particularly useful themselves as imaging contrast agents. Further, the inclusion of other components in multiple copies renders them useful in other respects as described herein. For instance, the inclusion of a chelating agent containing a paramagnetic ion makes the emulsion useful as an MRI contrast agent. The inclusion of biologically active materials makes them useful as drug delivery systems. The inclusion of radionuclides makes them useful either as therapeutic for radiation treatment or as diagnostics for imaging. Other imaging agents include fluorophores, such as fluorescein or dansyl. Biologically active agents may be included. A multiplicity of such activities may be included; thus, images can be obtained of targeted tissues at the same time active substances are delivered to them.

The emulsions can be prepared in a range of methods depending on the nature of the components to be included in the coating.

In one procedure, used for illustrative purposes only, the following procedure is set forth: perfluoroctylbromide (PFOB, 20% v/v), a surfactant co-mixture (1.5% w/v), glycerin (1.7% w/v) and water representing the balance is prepared where the surfactant co-mixture includes 97.9 mole % lecithin, 0.1 mole % vitronectin antagonist conjugated to PEG₂₀₀₀-phosphatidylethanolamine, and 1 mole % of a lipophilic chelate (Methoxy-DOTA-caproyl-phosphatidylethanolamine (MeO-DOTA-PE). The surfactant components are prepared as previously published (Lanza et al. (1996) Circulation 94:3334-40), combined with PFOB and distilled deionized water and emulsified at 20,000 PSI for four minutes. A drug can be added in titrated amounts between 0.01 and 50 mole % of the 2% surfactant layer, between 0.01 and 20 mole % of the 2% surfactant layer, between 0.01 and 10 mole % of the 2% surfactant layer, between 0.01 and 5.0 mole % of the 2% surfactant layer, preferably between 0.2 and 2.0 mole % of the 2% surfactant layer. The chloroform-lipid mixture is evaporated under reduced pressure, dried in a 50° C. vacuum oven overnight and dispersed into water by sonication. The suspension is transferred into a blender cup (for example, from Dynamics Corporation of America) with iodized oil in distilled or deionized water and emulsified for 30 to 60 seconds. The emulsified mixture is transferred to a Microfluidics emulsifier and continuously processed at 20,000 PSI for four minutes. The completed emulsion is vialed, blanketed with nitrogen and sealed with stopper crimp seal until use. A control emulsion can be prepared identically excluding the drug from the surfactant co-mixture. Particle sizes are determined in triplicate at 37° C. with a laser light scattering submicron particle size analyzer (Malvern Zetasizer 4, Malvern Instruments Ltd., Southborough, Mass.), which indicate tight and highly reproducible size distribution with average diameters less than 200 nm. Unincorporated drug can be removed by dialysis or ultrafiltration techniques. To provide the targeting ligand, for example, an antibody or antibody fragment or a non-peptide ligand is coupled covalently to the phosphatidyl ethanolamine through a bifunctional linker in the procedure described herein.

Kits

The emulsions of the invention may be prepared and used directly in the methods of the invention, or the components of the emulsions may be supplied in the form of kits. The kits may comprise the untargeted composition containing all of the desired ancillary materials in buffer or in lyophilized form. The kits may comprise the pre-prepared targeted composition containing all of the desired ancillary materials and targeting materials in buffer or in lyophilized form. Alternatively, the kits may include a form of the emulsion which lacks the targeting agent which is supplied separately. Under these circumstances, typically, the emulsion will contain a reactive group, such as a maleimide group, which, when the emulsion is mixed with the targeting agent, effects the binding of the targeting agent to the emulsion itself. A separate container may also provide additional reagents useful in effecting the coupling. Alternatively, the emulsion may contain reactive groups which bind to linkers coupled to the desired component to be supplied separately which itself contains a reactive group. A wide variety of approaches to constructing an appropriate kit may be envisioned. Individual components which make up the ultimate emulsion may thus be supplied in separate containers, or the kit may simply contain reagents for combination with other materials which are provided separately from the kit itself.

A non-exhaustive list of combinations might include: emulsion preparations that contain, in their lipid-surfactant layer, an ancillary component such as a fluorophore or chelating agent and reactive moieties for coupling to the targeting agent; the converse where the emulsion is coupled to targeting agent and contains reactive groups for coupling to an ancillary material; emulsions which contain both targeting agent and a chelating agent but wherein the metal to be chelated is either supplied in the kit or independently provided by the user; preparations of the nanoparticles comprising the surfactant/lipid layer where the materials in the lipid layer contain different reactive groups, one set of reactive groups for a targeted ligand and another set of reactive groups for an ancillary agent; preparation of emulsions containing any of the foregoing combinations where the reactive groups are supplied by a linking agent.

In one embodiment, the kit for the preparation of an emulsion of nanoparticles targeted to tissue expressing αᵥβ₃ comprises at least one container that contains nanoparticles comprising a ligand specific for αᵥβ₃ and a linking moiety for coupling to a low resolution contrast agent and/or a higher resolution contrast agent, at least one container that contains said low resolution contrast agent, and at least one container that contains said higher resolution contrast agent.

In another embodiment, the kit for the preparation of an emulsion of nanoparticles targeted to tissue expressing αᵥβ₃ comprises at least one container that contains nanoparticles comprising a linking moiety for coupling to a ligand specific for αᵥβ₃, at least one container that contains a ligand specific for αᵥβ₃, at least one container that contains a low resolution contrast agent, and at least one container that contains a higher resolution contrast agent.

The invention is also directed to a kit for high resolution imaging, comprising at least one container that contains nanoparticles comprising a ligand specific for αᵥβ₃ coupled via a linking moiety to a low resolution contrast agent, and at least one container that contains nanoparticles comprising a ligand specific for αᵥβ₃ coupled via a linking moiety to a higher resolution contrast agent.

In another embodiment, the kit for high resolution imaging comprises at least one container containing halocarbon-based nanoparticles comprising a ligand specific for a target moiety, wherein the nanoparticles are coupled to a higher resolution contrast agent.

The kits of the invention can further comprise instruction means for administering the contrast agents to a subject. The instruction means can be a written insert, an audiotape, an audiovisual tape, or any other means of instructing the administration of the contrast agents to a subject, whereby a target tissue is located using a low resolution imaging technique and further visualized using a higher resolution imaging technique.

The following examples are intended to illustrate but not to limit the invention.

### EXAMPLE 1

Preparation of αᵥβ₃-Targeted ¹¹¹In Nanoparticles

αᵥβ₃-Targeted ¹¹¹In perfluorocarbon nanoparticles were prepared by emulsification of 20% (v/v) perfluoroctylbromide, 1.5% (w/v) of a surfactant co-mixture, 1.7% (w/v) glycerin and water for the balance (Lanza et al. (1996) Circulation 94:3334-3340; Flacke et al. (2001) Circulation 104(11):1280-1285; Winter et al. (2003) Cancer Res. 63(18):5838-5843). The surfactant co-mixture generally included 97.9 mole % lecithin (Avanti Polar Lipids, Inc.), 0.1 mole % vitronectin antagonist conjugated to PEG₂₀₀₀-phosphatidylethanolamine (Avanti Polar Lipids, Inc.) (Winter et al. (2003) Cancer Res. 63(18):5838-5843), and 1 mole % of a lipophilic chelate (Methoxy-DOTA-caproyl-phosphatidylethanolamine (MeO-DOTA-PE), Dow Chemical Company) (Winter et al. (2005) J. Magn. Magn. Mater. 293 (1):540-545). The surfactant components were prepared as previously published (Lanza et al. (1996) Circulation 94:3334-3340), combined with PFOB and distilled deionized water and emulsified (Microfluidics, Inc.) at 20,000 PSI for four minutes.

Particle sizes were nominally 242 nm (polydispersity index of 0.231), determined at 37° C. with a laser light scattering submicron particle analyzer (Zetasizer 4, Malvem Instruments). Bioactivity of the αᵥβ₃-integrin targeted nanoparticles was confirmed using an in vitro vitronectin cell adhesion assay as previously reported (Schmieder et al. (2005) Magn. Reson. Med 53(3):621-627).

Efficient solid-phase coupling of multiple ¹¹¹In to nanoparticles proved difficult with direct coupling methods due to variable hydrolysis and precipitation of the metal. Several direct coupling labeling methods were conducted in 0.1 M ammonium acetate buffered solution pH 5.5, 0.2 M sodium carbonate solution, 0.2 M sodium hydroxide, or 10% v/v triethylamine buffer in combination with heating to 65° C. for 30 minutes with generally poor and variable results due to significant hydrolysis of the free metal. A TLC profile was obtained from the co-incubation of control emulsion (i.e., without homing ligand or DOTA) and ¹¹¹In followed by the addition of DTPA. Approximately 40% of the label remains at the origin (rf=0).

This problem was resolved utilizing citrate, a weak chelator, as a shuttle that transiently complexed with the ¹¹¹In and minimized hydrolysis. In the presence of 0.5 M sodium citrate, ¹¹¹In hydroxide precipitation was reduced to <2%. Subsequent addition of αᵥβ₃-integrin-targeted nanoparticles rich in surface methoxy-benzyl DOTA, a strong chelator, favorably competed the ¹¹¹In from the citrate, yielding more reproducible labeling.

Although coupling of ¹¹¹In to free DOTA chelate in solution was accomplished with essentially stoichiometric precision, the efficiency of solid-phase coupling of ¹¹¹In to methoxybenzylDOTA on the nanoparticles was poorer, despite a marked excess of surface chelate. Nevertheless, very high specific activity (˜10 ¹¹¹In/nanoparticle) was obtained routinely for this study.

Generally, 250 μl of 0.5 M sodium citrate pH 5.7 was combined with 40 MBq of ¹¹¹InCl₃ in 0.04 M HCl (250 μl). The indium-citrate buffer was mixed with αᵥβ₃-integrin-nanoparticles in ratios to produce particles with ˜1 or ˜10 nuclides each. Following overnight incubation in a ˜40° C. shaker bath (50 RPM), free DTPA was added to the reaction mixture for 5 minutes to scavenge the free radionuclide.

Coupling was assessed by thin layer chromatography (TLC) at ambient temperature. An aliquot of the above mixture was applied to silica gel coated paper and developed in 0.1 M ammonium acetate (pH 5.5):methanol:water (20:100:200, v/v). One cm strips were counted with an automatic gamma counter (Wizard 3″ model 1480, Perkin Elmer). Radioactive nanoparticle payload was calculated as the ratio of radioactivity per μl assessed by TLC associated with the nanoparticles to the number of particles/μl of emulsion based on their nominal size and perfluorocarbon concentration. Coupling efficiency of ¹¹¹In to the nanoparticles ranged from ˜50 to ˜70% for the high (10 nuclides/particle) and ˜85 to ˜90+% for 1 nuclide/particle formulations. Equivalent total dosages of nanoparticles among treatments were maintained by addition of unlabeled, nontargeted emulsion to the high specific activity injectate.

### EXAMPLE 2

### Pharmacokinetics of Radiolabeled Nanoparticles

Animals were maintained and physiologically monitored throughout these studies in accordance with protocol and procedures approved by the Animal Studies Committee at Washington University Medical School.

Basic pharmacokinetic parameters of radiolabeled nanoparticles were estimated in six New Zealand White rabbits administered αᵥβ₃-targeted ¹¹¹In nanoparticles (11 MBq/kg) bearing 10 ¹¹¹In/particle via ear vein bolus injection. Blood was sampled via a separate venous access at baseline and 2, 5, 10, 20, 30, 45, 60, 90, and 120 minutes following injection, weighed, counted in an automatic gamma well counter (Wizard 1480, Perkin Elmer), and the results normalized for slight volume differences. For each animal, a simple biexponential model, y=A₀e^{-at}+B₀e^{-bt}, was fit to the data, from which estimates of distribution volume, elimination rates, and clearance were derived using standard kinetic modeling equations for an open two compartment model (O'Flaherty E J. Toxicants and Drugs: kinetics and dynamics. New York: John Wiley & Sons, 1981).

All variables are presented as mean±standard error of the mean (SEM). General linear models including Student's t-tests and ANOVA using SAS (SAS Institute) were used for the analysis of continuous variables. Least significant difference methods (LSD) were used for means separation at an alpha level of 0.05.

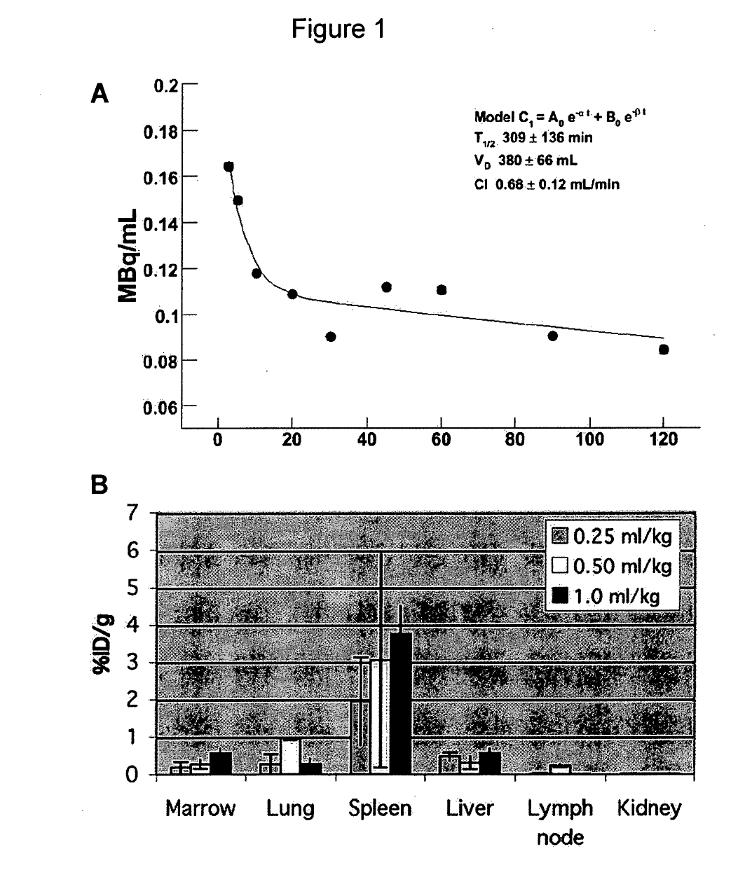

The pharmacokinetics of ¹¹¹In αᵥβ₃-nanoparticles (˜10 ¹¹¹n/NP) were defined in six rabbits. FIG. 1a illustrates a two compartment modeling of the data from one rabbit over the initial two hours. Based upon the coefficients and rate estimates derived from these data, the beta elimination half-life (t_{1/2β}) of the nanoparticles was estimated to be 309 min±136 min (SD). The volume of distribution (V_{D}) and clearance (Cl) were calculated to be 380 ml±66 ml (SD) and 0.68 ml/min±0.12 ml/min (SD), respectively, in these young rabbits. The data suggest that perfluorocarbon nanoparticles exhibit sufficient circulatory half-life that is more than adequate to reach and saturate any vascular receptor. The volume of distribution was approximately twice as large as estimates of the circulatory volume, reflecting uptake and clearance by the reticuloendothelial system.

### EXAMPLE 3

### Biodistribution of αᵥβ₃-Targeted ¹¹¹In Nanoparticles

The biodistribution of αᵥβ₃-targeted perfluorocarbon nanoparticles was determined three-hours post injection in New Zealand White rabbits randomly administered intravenous dosages of 0.25 ml/kg (n=3), 0.5 mg/kg (n=3) and 1.0 ml/kg (n=3). Rabbits were euthanized and the primary particular clearance organs (i.e., lung, spleen, liver, lymph node, bone marrow, kidney) were excised, weighted and prepared for perfluorocarbon analysis.

Perfluorocarbon concentration was determined with gas chromatography using flame ionization detection (Model 6890, Agilent Technologies, Inc. Wilmington, Del.). Weighed tissue aliquots were extracted in 10% potassium hydroxide in ethanol. Two ml of internal standard (0.1% octane in Freon) was added, and the mixture was sealed in a serum vial. The sealed vial contents were vigorously vortexed then continuously agitated on a shaker for 30 minutes. The lower extracted layer was filtered through a silica gel column and stored at 4-6° C. for analysis. Initial GC column temperature was 30° C. and ramped upward at 10° C./minute to 145° C. All samples were assayed in duplicate and the results were expressed as % ID/g±SD.

As shown in FIG. 1b, perfluorocarbon content was greatest in the spleen as % ID/g tissue, with concentrations increasing from 1.0±1.1% ID/g, 3.0±2.8% ID/g, and 3.7±0.8% ID/g for the 0.25 ml/kg, 0.5 ml/kg, and 1.0 ml/kg emulsion dosages, respectively. At the 1.0 ml/kg emulsion dosage level, liver perfluorocarbon content was 15% (0.6±0.1% ID/g) of that measured in the spleen. In general, the perfluorocarbon concentrations of the remaining tissues were less.

### EXAMPLE 4

### Targeting Tumors using αᵥβ₃-Targeted ¹¹¹In Nanoparticles

Male New Zealand White Rabbits (˜2.5 kg) were anesthetized with intramuscular ketamine and xylazine (65 and 13 mg/kg, respectively). The left hind leg of each animal was shaved, sterile prepped, and infiltrated locally with Marcaine™ prior to placement of a small incision above the popliteal fossa. A 2 by 2 mm Vx-2 carcinoma tumor fragment (NCI tumor depository) was freshly obtained from a donor animal and implanted at a depth of approximately 0.5 cm within the fossa. Anatomical planes were approximated and secured with a single absorbable suture, and the skin incision was sealed with Dermabond™ skin glue. Following the tumor implantation procedure, the effects of xylazine were reversed with yohimbine, and animals were allowed to recover.

Twelve to 16 days after Vx-2 implantation rabbits were anesthetized with 1% to 2% Isoflurane™, intubated, ventilated, and positioned 3 cm below the high energy pinhole collimator equipped with a single 3 mm aperture and mounted to the clinical Genesys gamma camera (Philips Medical Systems) operating in planar mode. Intravenous and intraarterial catheters, were placed in opposite ears of each rabbit, and used for systemic injection of nanoparticles and arterial blood sampling/physiologic monitoring. Dosages of labeled nanoparticles were calibrated for activity immediately prior to use with a Capintec CRC-15R well counter.

In vivo detection of angiogenesis in ˜12d Vx-2 tumors was studied in 16 New Zealand rabbits, which were randomized to receive 22 MBq/kg of either: 1) αᵥβ₃-integrin-targeted NP with ˜10 ¹¹¹In/NP (n=3); 2) αᵥβ₃-integrin-targeted NP with ˜1 ¹¹¹In/NP (n=4); 3) αᵥβ₃-integrin-targeted non-radioactive NP given (3:1) with αᵥβ₃-integrin targeted nanoparticles with ˜10 ¹¹¹In/NP (i.e., competition group, n=3); 4) non-targeted NP with ˜10 ¹¹¹In/NP (n=3); or 5) non-targeted NP with ˜1 ¹¹¹In/NP (n=3).

Following intravenous injection, dynamic nuclear images (matrix:128×128) were acquired using two 20% windows centered at 170 keV and 244 keV at baseline and serially, every 15 minutes for two hours. DICOM images were exported to a Unix workstation and later analyzed with ImageJ software (NIH.gov). Anatomical landmarks were identified on each frame and regions-of-interest (ROI) of comparable size were manually placed around the tumor signal, muscle, and background regions to determine average pixel activity.

An additional eight rabbits with Vx-2 tumors were administered either αᵥβ₃-integrin-targeted (n=4) or nontargeted (n=4) NP with ˜10 ¹¹¹In/NP and imaged at 18 hours (n=4) or 48 hours (n=4). At 18 hours, rabbits were scanned dynamically every 15 minutes for 2 hours. At 48 hours, one 15-minute image acquisition was performed.

After imaging, animals were euthanized and tumors resected, weighed and fixed in formalin or quickly frozen in OCT for routine histopathology and selective immunohistochemistry. In two animals, testicles were excised as a positive control to confirm neovascularity, which develops continuously in the spermatic cords. Acetone-fixed, frozen tissues were sectioned (5 μm) and routinely stained with hematoxylin and eosin and or immunostained for αᵥβ₃-integrin (LM-609, Chemicon International, Inc). Immunohistochemistry was performed using the Vectastain® Elite ABC kit (Vector Laboratories), developed with the Vector® VIP kit. Microscopic images were obtained using a Nikon E800 research microscope and digitized with a Nikon DXM1200 camera.

In a separate cohort of animals (n=2), αᵥβ₃-targeted nanoparticles (0.1 ml/kg) labeled with rhodamine and FITC-lectin (Vector Laboratories), a general stain for vascular endothelium, were administered intravenously. The αᵥβ₃-targeted rhodamine nanoparticles (0.1 ml/kg) were give two hours before the FITC-lectin, in concert with nuclear imaging protocol, and the fluorescent lectin was given about 15 minutes before euthanasia. Rabbits were extensively perfused with saline before tissue extraction to remove unbound fluorescent labels, before embedding the tumors in OCT for frozen sectioning and microscopy.

All variables are presented as mean±standard error of the mean (SEM). General linear models including Student's t-tests and ANOVA using SAS (SAS Institute) were used for the analysis of continuous variables. Least significant difference methods (LSD) were used for means separation at an alpha level of 0.05.

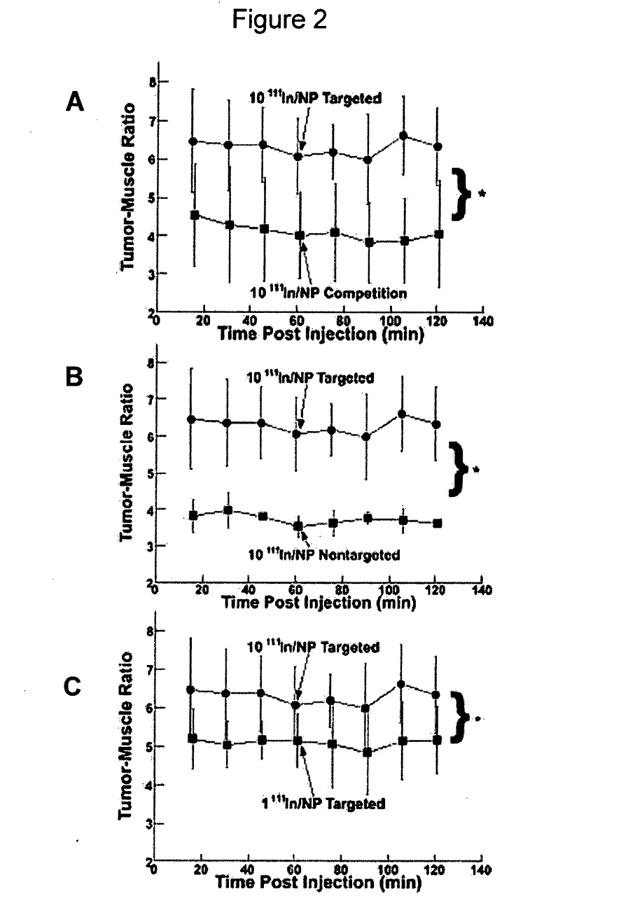

Dynamic imaging was conducted for two hours post intravenous injection and the tumor-to-muscle ratio of mean pixel intensity in rabbits given ¹¹¹In αᵥβ₃-nanoparticles bearing ˜10 ¹¹¹In/NP was compared to animals receiving. ¹¹¹In αᵥβ₃-nanoparticles with a 3-fold competitive dosage of nonlabeled αᵥβ₃-nanoparticles (FIG. 2a). ¹¹¹In αᵥβ₃-nanoparticles produced high tumor-to muscle ratio (TMR) contrast (6.46±0.78) within 15 minutes of injection, which persisted throughout the two-hour period and averaged 6.3±0.07. Blockade of integrin receptors with nonlabeled αᵥβ₃-nanoparticles lowered the TMR contrast at 15 minutes to 4.53±0.77, and this difference persisted over the two hours of serial imaging, averaging 4.11±0.08 (p<0.05). Nontargeted ¹¹¹In nanoparticles (FIG. 2b) demonstrated lower TMR contrast at 15 minutes (3.82±0.32) and over two hours (3.74±0.05) than did the integrin-targeted formulation (p<0.05). The tumor contrast response of the nontargeted and competition treatments did not differ (p>0.05). At two hours, the percent injected dose (% ID) at the tumor site of rabbits administered ¹¹¹In αᵥβ₃-nanoparticles was 1.20% ID±0.18% ID, which was higher than the dosage retained in animals receiving the equivalent nontargeted nanoparticles, 0.60% ID±0.08% ID (p<0.05). Collectively, these results support the superior contrast enhancement obtained with αᵥβ₃-integrin targeting, and suggest that passive targeting of the neovasculature may contribute significantly to the initial overall tumor-to-muscle contrast ratio.

In FIG. 2c, signal enhancement relative to muscle of ¹¹¹In αᵥβ₃-nanoparticles with ˜10 ¹¹¹In/NP was superior (p<0.05) over two hours to particles formulated with ˜1 ¹¹¹In/NP (5.09±0.04). However, the average contrast achieved with the lower activity agent was not different (p>0.05) from the signal obtained with a nontargeted formulation bearing ˜1 ¹¹¹In/NP (data not shown).

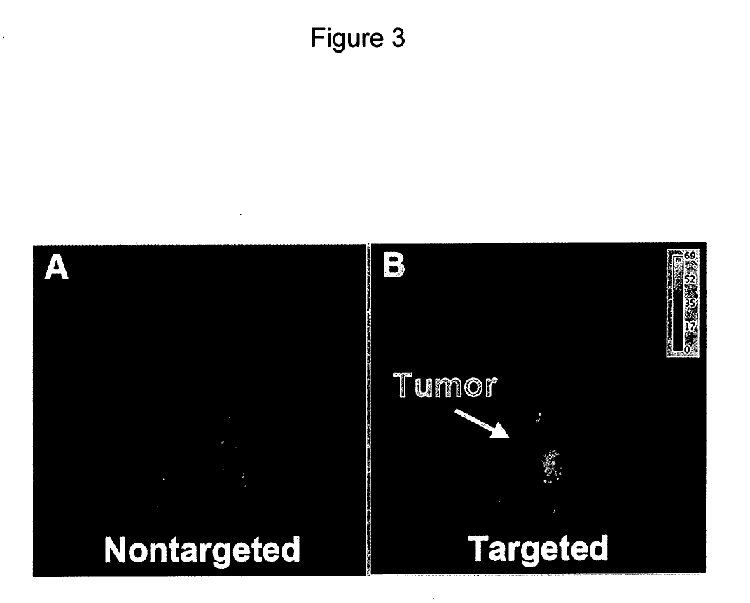

Another cohort of eight rabbits was examined after 18 hours (˜3 circulating half-lives) and 48 hours (˜8 circulating half-lives) to assess the persistence of the targeted nuclear signal. FIG. 3 illustrates 18-hour images of two rabbits (one targeted, FIG. 3b, and one control, FIG. 3a ), which received equivalent radioactive dosages of ¹¹¹In nanoparticles and exhibited similar muscle background counts. The contrast of the integrin-targeted formulation was greater than that of the non-targeted agent. For animals receiving ¹¹¹In αᵥβ₃-nanoparticles, the average percent injected dose at the tumor site was four times greater (p<0.05; 0.48%ID±0.04%ID) than that left in animals receiving the nontargeted control (0.10%ID±0.04%ID/kg). At 48 hours post-injection, the signal from tumor and muscle were substantially lower and indistinguishable between groups (p>0.05).

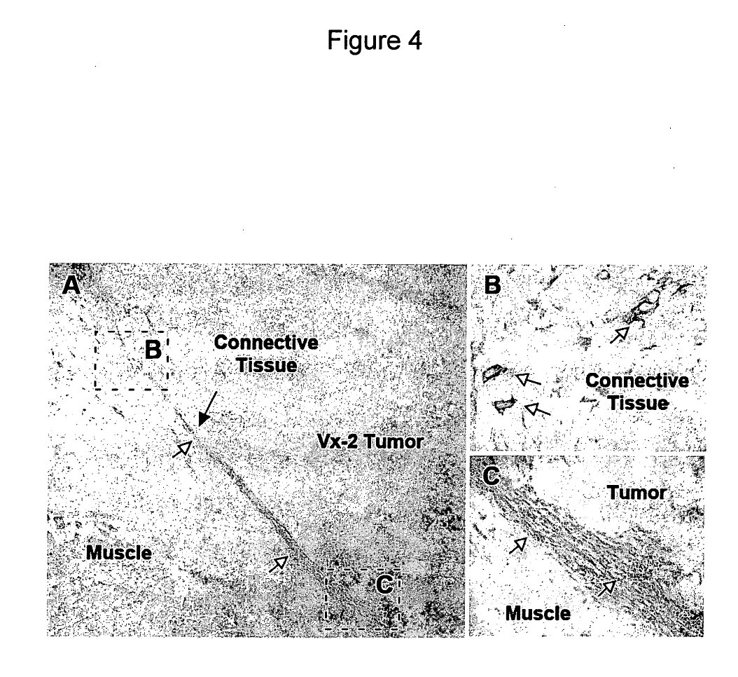

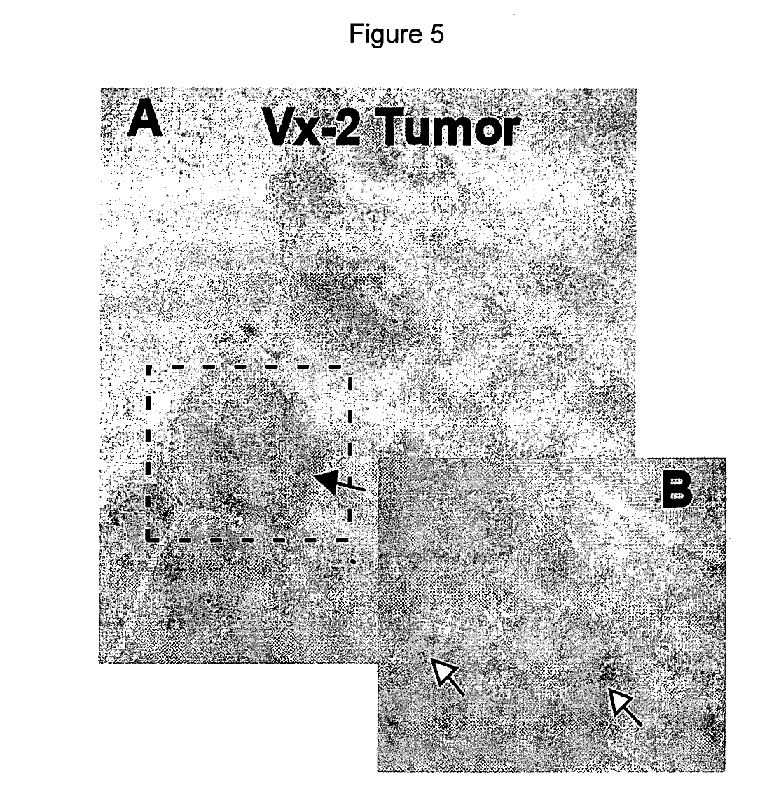

Histological analysis of αᵥβ₃-integrin expression revealed that the expression of αᵥβ₃-integrin occurred asymmetrically along tissue interfaces between tumor and adjacent vascular structures within connective tissue fascia and vessel adventia. The up-regulated expression of αᵥβ₃-integrin extended beyond the tumor capsule and was recognized in nearby vascular structures associated with muscle fascia (FIGS. 4a-c). The αᵥβ₃-integrin vascular expression was also detected in other organs including maturing testicular epididymis (as confirmed by histology) and in the epiphyseal growth plate region of the femur and tibia. Macrophages, an abundant source of αᵥβ₃-integrin were identified with RAM-11 staining and found densely distributed within the tumor core (FIGS. 5a and b) but only sparsely in connective tissue surrounding the tumor.

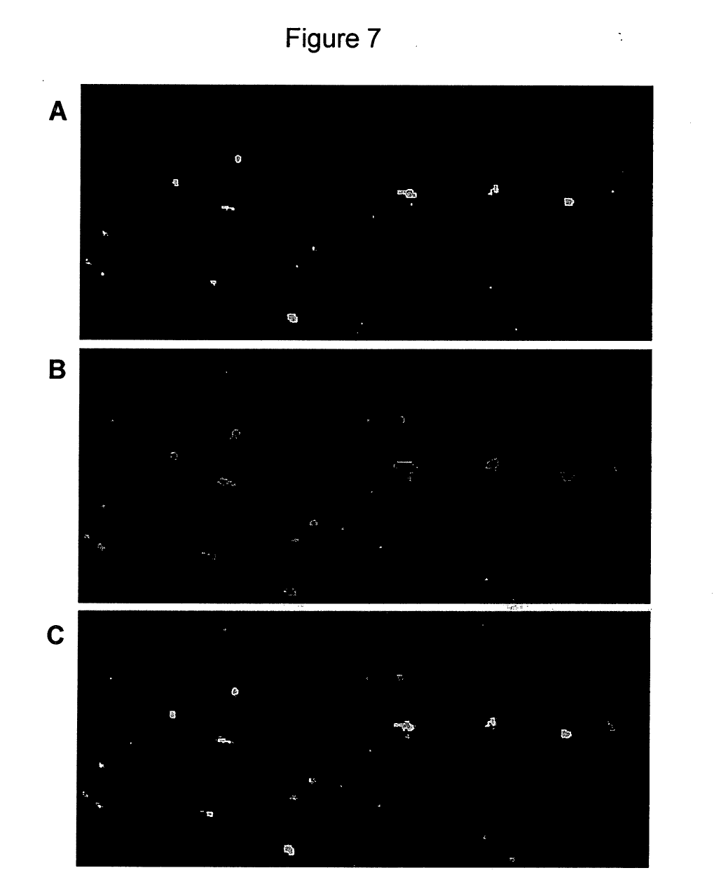

Intravenous co-administration of αᵥβ₃-targeted rhodamine nanoparticles and FITC-lectin, a vascular endothelial marker, revealed a close spatial correlation between the two markers. FITC-lectin was found throughout the vasculature including the neovessels as shown in FIGS. 7A-C. Rhodamine nanoparticles were predominantly located in the smaller vessels and co-localized with the FITC-lectin.

### EXAMPLE 5

### Preparation of αᵥβ₃-Targeted Fluorescent Nanoparticles

Fluorescent nanoparticles were prepared by incorporating AlexaFluor 488 coupled to caproyl-phosphatidylethanolamine into the surfactant at 0.5 mole %. AlexaFluor 488-caproyl-phosphatidylethanolamine was synthesized by dissolving 7.8 μmole AlexaFluor 488 carboxylic succinimidyl ester (Molecular Probes) in 1.4 ml dimethylformamide and mixing it with 10 μmole caproylamine phosphatidylethanolamine (Avanti Polar Lipids) in 200 μl chloroform at 37° C. for one hour. Following addition of 200 μl of chloroform, reaction temperature was increased to 50° C. and continued overnight.

TLC using a reverse phase hydrocarbon (C₁₈) impregnated silica gel and a mobile phase consisting of 0.1 M sodium acetate buffer (pH 5.6):methanol:water at a ratio of 20:100:200 was performed to monitor and purify the conjugated product from the uncoupled AlexaFluor dye. The red fluorescent lipid was recovered at the origin, extracted with chloroform:methanol (3:1), and evaporated to dryness until use.

Microscopic localization of nanoparticles within and around the tumor was studied in a separate cohort of Vx-2 implanted rabbits (n=2), which received αᵥβ₃-targeted nanoparticles (0.1 ml/kg) with AlexaFluor 488 cyan dye incorporated into the surfactant. The fluorescent nanoparticles were administered with a 10-fold excess of non-targeted, non-labeled nanoparticles to minimize passive accumulation within the neovasculature and allowed one hour to circulate. Animals were killed, and the tumor was removed, rinsed repeatedly in phosphate buffered saline, and frozen in OCT medium. Frozen tumor sections (4 μm) were counterstained with DAPI to identify nuclei. Photomicrographs of green AlexaFluor nanoparticles and DAPI-labeled nuclei were superimposed to assess the distribution of the contrast agent with respect to other cellular elements. Adjacent sections were stained with RAM-11 (Dako, Inc.) to delineate macrophage distribution within the tumor.

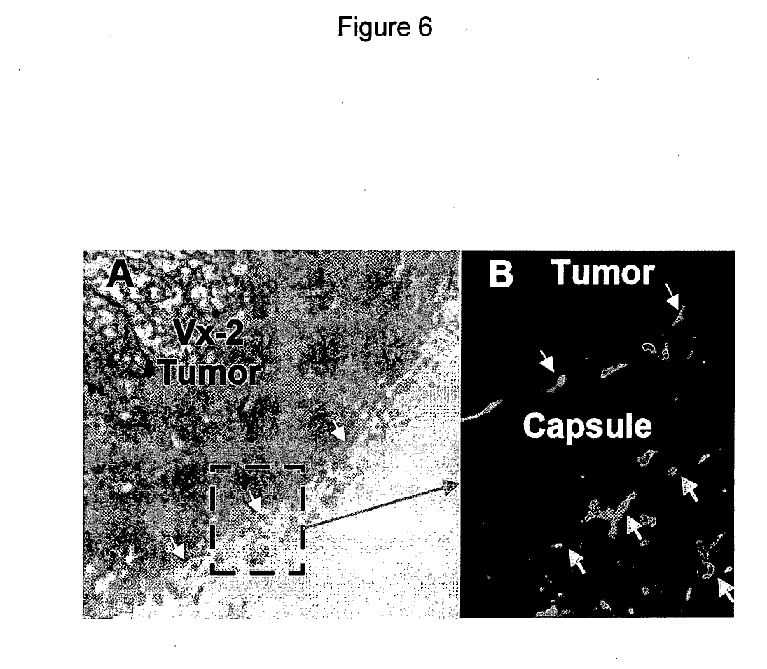

Fluorescence microscopy of frozen tumor tissues showed that the AlexaFluor particles were within the capsular interface between adjacent muscle (FIG. 6a), corresponding to the distribution of αᵥβ₃-integrin positive vessels (FIG. 6b). Immunohistological co-staining of αᵥβ₃-integrin positive vessels with LM609 in rabbits pretreated with αᵥβ₃-targeted AlexaFluor 488 nanoparticles was competitively inhibited by the receptor by bound particles. The distribution of αᵥβ₃-targeted AlexaFluor 488 nanoparticles was not associated with macrophages stained by RAM 11.

In summary, αᵥβ₃-targeted ¹¹¹In nanoparticles were developed and studied for use as sensitive beacons of angiogenesis in nascent tumors. Tumor neovasculature was rapidly identified with the targeted nanoparticles, but blood pool persistence and slow washout of passively entrapped nanoparticles required overnight delays for clearance to occur. The results suggest that αᵥβ₃-targeted ¹¹¹In nanoparticles may provide a clinically robust and rapid beacon for detecting angiogenesis in vivo, which could augment efforts to identify and treat tumors early.

Therefore, the low resolution signal from radiolabeled nanoparticles in the tumor neovasculature can be used to rapidly identify potential regions-of-interest and guide high-resolution, secondary imaging, such as MR or CT imaging. Moreover, the particles could be used alone at minimal dosages to localize sites of interest and followed by noncontrast-enhanced imaging or αᵥβ₃-nanoparticles with or without a paramagnetic label for ¹H and or ¹⁹F, respectively.

1. A method for high resolution imaging, comprising
a) administering a targeted low resolution contrast agent and a targeted higher resolution contrast agent having an analogous target as the low resolution contrast agent to a subject, and allowing each contrast agent to accumulate in a target tissue;
b) identifying the target tissue using a low resolution imaging technique to localize an accumulation of the low resolution contrast agent; and
c) obtaining a high resolution image of the target tissue using a high resolution imaging technique to localize an accumulation of the higher resolution contrast agent, thereby allowing the generation of a higher resolution image than that obtained by the use of the low resolution contrast agent alone.

2. The method of claim 1, wherein the low resolution contrast agent and the higher resolution contrast agent are the same agent detectable using a low resolution modality and a higher resolution modality.

3. The method of claim 1, wherein a decoy particle is administered with the low resolution contrast agent.

4. The method of claim 1, wherein the low resolution contrast agent and/or higher resolution contrast agent is incorporated into a nanoparticle.

5. The method of claim 4, wherein the low resolution contrast agent and higher resolution contrast agent are incorporated into the same nanoparticle.

6. The method of claim 4, wherein the nanoparticle is contained within an emulsion.

7. The method of claim 6, wherein the emulsion of nanoparticles comprises a liquid halocarbon core surrounded by a lipid coating.

8. The method of claim 1, wherein the low resolution and/or higher resolution contrast agent is targeted by a target-specific ligand.

9. The method of claim 8, wherein the target-specific ligand is an antibody, an antibody fragment, a peptide, an aptamer, a peptide mimetic, a drug or a hormone.

10. A method of delivering targeted contrast agents to a target tissue, which method comprises
a) administering a low resolution targeted contrast agent to a subject comprising a target tissue;
b) administering a higher resolution targeted contrast agent to the subject, wherein the higher resolution contrast agent has an analogous target as the low resolution contrast agent; and
c) allowing the contrast agents to accumulate in the target tissue, to thereby deliver targeted contrast agents to the target tissue.

11. The method of claim 10, wherein the low resolution and/or higher resolution contrast agent is incorporated into a nanoparticle.

12. The method of claim 11, where the low resolution and higher resolution contrast agents are incorporated into the same nanoparticle.

13. The method of claim 11, wherein the nanoparticle is contained within an emulsion.

14. The method of claim 13, wherein the emulsion of nanoparticles comprises a liquid halocarbon core surrounded by a lipid coating.

15. The method of claim 10, further comprising obtaining an image of the targeted tissue bound to the low resolution contrast agent.

16. The method of claim 15, further comprising obtaining a high resolution contrast image of the targeted tissue.

17. The method of claim 10, wherein the target tissue is characterized by high levels of αᵥβ₃ integrin, and wherein the low resolution and/or higher resolution contrast agent is coupled to a ligand for αᵥβ₃ integrin.

18. A kit for the preparation of an emulsion of nanoparticles targeted to a tissue expressing a target moiety, which kit comprises at least one container that contains nanoparticles comprising a ligand specific for the target moiety and a linking moiety for coupling to a low resolution contrast agent and/or a higher resolution contrast agent, at least one container that contains the low resolution contrast agent, and at least one container that contains the higher resolution contrast agent.

19. The kit of claim 18, wherein the nanoparticles are halocarbon-based nanoparticles that further comprise a coating of lipid/surfactant.

20. The kit of claim 18, wherein the target moiety is αᵥβ₃.

21. The kit of claim 18, wherein the higher resolution contrast agent comprises at least one MRI contrast agent.

22. The kit of claim 18, wherein the low resolution contrast agent comprises ^{99m}Tc.

23. A kit for the preparation of an emulsion of nanoparticles targeted to a tissue expressing a target moiety, which kit comprises at least one container that contains nanoparticles comprising a linking moiety for coupling to a ligand specific for the target moiety, at least one container that contains a ligand specific for the target moiety, at least one container that contains a low resolution contrast agent, and at least one container that contains a higher resolution contrast agent.

24. A kit for high resolution imaging, comprising at least one container that contains a targeted low resolution contrast agent, at least one container that contains a higher resolution contrast agent, and instructions means for use.

25. The kit of claim 24, wherein one or both of the contrast agents is targeted to αᵥβ₃.

26. The kit of claim 25, wherein the high resolution contrast agent is selected from the group consisting of an MRI agent, a CT imaging agent, an optical imaging agent, an ultrasound imaging agent, a paraCEST imaging agent, and a combination thereof.

27. A method to obtain a magnetic resonance image of a target, wherein the higher resolution contrast agent comprises an MRI agent, which method comprises administering the composition of claim 25 to the target; and obtaining a magnetic resonance image of the target.

28. The method of claim 27, wherein the target is contained in a mammalian subject.

29. The kit of claim 25, wherein one or both of the contrast agents comprise nanoparticles.

30. A kit for high resolution imaging, comprising at least one container that contains halocarbon-based nanoparticles comprising a ligand specific for a target moiety, wherein the nanoparticles are coupled to a higher resolution contrast agent, and instructions means for use.

31. The kit of claim 30, wherein the halocarbon-based nanoparticles comprise PFOB.

32. A method to obtain a high resolution image of a target tissue, wherein the higher resolution contrast agent comprises a MRI contrast agent, which method comprises administering the composition of claim 31 to a subject;
identifying a target tissue using a fluorine MRI to localize an accumulation of the low resolution contrast agent in the target tissue; and
obtaining a magnetic resonance image of the target tissue, thus generating a high resolution image of the target tissue.
